(19) United States
(12) Patent Application Publication
(10) Pub. No.: US 2025/0226097 A1
(43) Pub. Date: Jul. 10, 2025

(54) Systems and Methods for Generating Dental Images and Animations to Assist in Understanding Dental Disease or Pathology as Part of Developing a Treatment Plan

(71) Applicant: Adra Corporation, Atlanta, GA (US)

(72) Inventors: Hamed Fesharaki, Atlanta, GA (US); Liushifeng Chen, Uppsala (SE)

(21) Appl. No.: 19/012,403

(22) Filed: Jan. 7, 2025

Related U.S. Application Data
(60) Provisional application No. 63/619,464, filed on Jan. 10, 2024.

(51) Int. Cl. G16H 50/20; A61C 13/34; G06T 7/00; G06T 13/80; G06V 20/70; G16H 10/60

(52) U.S. Cl. CPC G16H 50/20; A61C 13/34; G06T 7/0016; G06T 13/80; G16H 10/60; G06T 2207/10116; G06T 2207/20081; G06T 2207/20084; G06T 2207/30008; G06T 2207/30036; G06T 2210/41; G06V 20/70; G06V 2201/03

(57) ABSTRACT

Systems, apparatuses, and methods to generate and present personalized animations based on patient dental records and information. Embodiments obtain dental images and related data for at least one dental object and use machine learning models to detect features including pathological, non-pathological, bone levels and anatomical structure. The data is used to generate a visual representation of the natural progression of one or more dental pathologies and the corresponding relationship(s) with the anatomy of the tooth. By illustrating the progression through visual means, the approach may be used to show how untreated diseases can impact different anatomical features, and thereby emphasize the importance of timely treatment.

### CROSS REFERENCE TO RELATED APPLICATION

This application claims the benefit of U.S. Provisional Application No. 63/619,464, filed Jan. 10, 2024, entitled “Systems and Methods for Generating Dental Images and Animations to Assist in Understanding Dental Disease or Pathology as Part of Developing a Treatment Plan”, the disclosure of which is incorporated, in its entirety (including the Appendix) by this reference.

### BACKGROUND

General dentists (and specialists) employ a variety of tools for diagnostic purposes, with dental radiographs and intraoral images serving as primary sources of information about a patient's teeth, gums, and oral health. Dental practitioners use x-ray radiographs to examine dental anatomy and to determine an appropriate treatment strategy and plan for the patient. Dental radiographs and digital images have been used by dentists for purposes of diagnosis, to find abnormalities, and to monitor the progress of a treatment (see I Ahmad. (2009). Digital dental photography. Part 2: Purposes and uses. National Library of Medicine, 9; 206 (9): 459-64).

Following examination of these images (i.e., x-rays, intraoral images, or other types of images or data), dentists analyze and diagnose the issues present in the patient's mouth, teeth, and gums. Due to the specialized nature of reading x-rays or intraoral images, interpretations of the images rely almost solely on the dentist's experience and expertise (see Wang, et al. (2016) A benchmark for comparison of dental radiography analysis algorithms. Medical Image Analysis, Vol. 31, pgs. 63-76).

Communicating a diagnosis and proposed treatment plan effectively to a patient depends to some extent on the dentist's personal experience and may pose a challenge, as dental radiographs and intraoral pictures are generally not comprehensible to those without experience and professional expertise (see Michelle Budd (2022) Reducing Noise in Dentistry: The Role of Al in Improving Radiographic Interpretation. Oral Health Group. Article retrieved 7 Dec. 2023). However, effectively communicating a diagnosis to a patient and ensuring their understanding of their dental issues and possible treatments is a crucial step in planning and executing a treatment plan. Given that most patients lack the expertise to interpret dental X-rays or intraoral images (which are the key diagnostic tools), this may create an obstacle and make it more difficult for a patient to comprehend their dental problem(s) and the possible consequences of not treating those problems.

As a result, this situation may cause a patient to delay in developing trust in the dentist's expertise, which is essential for patients to accept a proposed treatment. The potential lack of trust arising from a lack of understanding has been a factor in patients (by some estimates, as much as 41%) seeking a second opinion to clarify their uncertainty regarding a diagnosis or proposed treatment (see Obtaining a second opinion is a neglected source of health care inequalities. Isr J Health Policy Res. V.8). Studies suggest that dentists can enhance patients' acceptance of their proposed treatments by employing various tools, with a primary focus on educational resources to help patients better comprehend their dental issues and proposed treatments.

Embodiments of the disclosure are directed to overcoming the disadvantages of conventional approaches to informing patients about their dental conditions and proposed treatment plans.

### SUMMARY

The terms “invention,” “the invention,” “this invention,” “the present invention,” “the present disclosure,” or “the disclosure” as used herein refer broadly to all subject matter disclosed and/or described in this document, the drawings or figures, and to the claims. Statements containing these terms do not limit the subject matter disclosed or the meaning or scope of the claims. Embodiments of this disclosure are defined by the claims and not by this summary. This summary is a high-level overview of various aspects of the disclosure and introduces some of the concepts that are further described in the Detailed Description section below. This summary is not intended to identify key, essential or required features of the claimed subject matter, nor is it intended to be used in isolation to determine the scope of the claimed subject matter. The subject matter should be understood by reference to appropriate portions of the entire specification, to any or all figures or drawings, and to each claim.

In some embodiments, the disclosure is directed to a system and associated methods for assisting dental service providers (dentists, dental assistants, and hygienists as non-limiting examples) to more effectively deliver services and educate patients. In one embodiment, this is accomplished by generating images and/or animations to assist a patient to better understand a dental disease or pathology as part of developing and monitoring a treatment plan. The disclosed and/or described processes, methods, operations, and functions may be implemented in the form of an application and/or a set of services provided through a platform or system. The application or services provide multiple features and functions to assist dental service providers as well as educate patients.

In one embodiment, the disclosed and/or described approach identifies dental pathologies (such as caries, periapical radiolucency, calculus, and furcation), non-pathologies (such as past restorative treatments, wisdom tooth removal, and inferior alveolar nerve treatment), dental anatomical structures (such as dentin, enamel, and pulp), and bone levels shown on dental radiographs or other images. Past restorative treatments may include fillings, root canal treatments, crowns, pontics, or implants, and are identified and in some cases imaged and/or measured. This information is used by a dentist in planning the course of treatment for a patient. These features also serve as a tool to educate a patient by leveraging image processing and other techniques to generate images and/or animations illustrating the possible progression of a dental condition if left untreated or to illustrate scenarios of one or more treatment plans.

The disclosed and/or described image processing techniques recognize patterns and anomalies in dental radiographs, which aids in planning the appropriate treatment course for a patient. The techniques generate a visual representation of the natural and expected progression of one or more dental pathologies and the corresponding relationship(s) with the anatomy of an affected tooth. By illustrating the progression through visual means, the approach may be used to show how untreated diseases can impact different anatomical features, and thereby emphasize the importance of a patient obtaining treatment in a timely manner.

In one embodiment, the disclosed system and associated method may include the following elements, components, functions, processes, or operations:
Capture or access one or more x-rays, intraoral images, or other types of images of a patient's mouth, teeth, and gums;
Classify the type of x-ray or image (i.e., the image mode and/or subject of the image);
Use one or more image processing techniques (such as a trained model or models) to identify, classify, or otherwise determine the following in the x-rays or images;
   One or more of the type, location, size, or dimensions of dental pathologies and non-pathologies (such as previous restorative treatments);
      The potential severity of a pathology;
   Tooth numbers or other accepted identifier;
   Bone level measurements;
   Anatomical tooth structures;
Generate one or more images or animations illustrating a likely progression over time of an untreated pathology for that patient;
   This includes utilizing the information obtained from the processing of the images for the specific patient;
   This may include accessing and using other patient-specific health related or dental information; and
Generate one or more images or animations illustrating a likely outcome of a proposed treatment plan for that patient;
   This includes utilizing the information obtained from the processing of the images for the specific patient;
   This typically will also include information regarding the specific treatment plan, its stages, timeline, and components.

In one embodiment, the disclosure is directed to a system for assisting dental service providers in their practice as well as educating patients by generating dental images and/or animations to assist a patient's understanding of a dental problem or pathology as part of developing a treatment plan. The system may include a set of computer-executable instructions, a memory or data storage element (such as a non-transitory computer-readable medium) on (or in) which the instructions are stored, and one or more electronic processors or co-processors. When executed by the processors or co-processors, the instructions cause the processors or co-processors (or a device of which they are part) to perform a set of operations that implement an embodiment of the disclosed and/or described method or methods.

In one embodiment, the disclosure is directed to a non-transitory computer readable medium containing a set of computer-executable instructions, wherein when the set of instructions are executed by one or more electronic processors or co-processors, the processors or co-processors (or a device of which they are part) perform a set of operations that implement an embodiment of the disclosed and/or described method or methods.

In some embodiments, the systems and methods disclosed and/or described herein may be implemented as a set of services or functionality provided through a SaaS or multi-tenant platform. The platform provides access to multiple entities, each with a separate account and associated data storage. Each account may correspond to a dentist, a hygienist, a dental assistant, an insurance company, an analytics company, a dental network, a group of dentists, or an organization, for example. Each account may access one or more services, a set of which are instantiated in their account, and which implement one or more of the methods or functions disclosed and/or described herein.

In one embodiment, the disclosed and/or described image processing technique(s) may be implemented as a backend service on a SaaS platform that provides other services to accounts residing on the platform. In such an example implementation, the operator of the SaaS platform or other form of system may implement the disclosed image processing technique(s) while providing other services or access to other applications for accounts on the platform.

Other objects and advantages of the systems, apparatuses, and methods disclosed and/or described will be apparent to one of ordinary skill in the art upon review of the detailed description and the included figures. Throughout the drawings, identical reference characters and descriptions indicate similar, but not necessarily identical, elements. While the embodiments disclosed and/or described herein are susceptible to various modifications and alternative forms, specific embodiments are shown by way of example in the drawings and are described in detail herein. However, embodiments of the disclosure are not limited to the specific or exemplary forms described. Rather, the disclosure covers all modifications, equivalents, and alternatives falling within the scope of the appended claims.

### BRIEF DESCRIPTION OF THE DRAWINGS

Embodiments of the disclosure are described with reference to the drawings, in which:

FIG. 1(a) is a diagram illustrating an example of the conventional workflow of a dental service provider compared to a corresponding workflow of a dental service provider using an embodiment of the disclosed approach (referred to as Adravision or the ADRA platform/system herein);

FIG. 1(b) is a flow chart or flow diagram illustrating the primary elements, components, and processes that may be implemented by a method to generate dental images and animations to educate patients by improving their understanding of a dental disease or pathology and aid in the development of a treatment, in accordance with an embodiment of the disclosure;

FIG. 1(c) is a second flow chart or flow diagram illustrating the primary elements, components, and processes that may be implemented by a method for educating patients by generating dental images and animations to assist in understanding a dental disease or pathology as part of developing a treatment plan, in accordance with an embodiment of the disclosure;

FIG. 2(a) is a diagram illustrating elements or components that may be present in a computer device or system configured to implement a method, process, function, or operation in accordance with an embodiment of the disclosure;

FIG. 2(b) is a chart illustrating treatment options to a patient and how delaying treatment can result in increased financial cost over time;

FIG. 2(c) is a diagram illustrating the elements, components, and processes in an example embodiment of the disclosed and/or described system and architecture;

FIG. 2(d) is a diagram illustrating a set of services or processes that may be implemented by a model or models (along with their respective outputs) that are made available through the system architecture of FIG. 2(c) in an example embodiment of the disclosed and/or described system and architecture;

FIG. 2(e) is a diagram illustrating a set of services or processes that may be implemented to determine a desirable treatment plan in an example embodiment of the disclosed and/or described system and architecture;

FIG. 2(f) is a flow chart or flow diagram illustrating a set of services or processes that may be implemented to compare one or more dental images with other available patient records, in accordance with an example embodiment of the disclosed and/or described system and architecture;

FIG. 2(g) is a diagram illustrating a set of services or processes that may be implemented to generate an animation of a scenario of disease progression and treatment options in an example embodiment of the disclosed and/or described system and architecture;

FIG. 2(h) is a diagram illustrating an example training process for a model that may be used in implementing an embodiment of the disclosure;

FIGS. 3-5 are diagrams illustrating a deployment of the system and methods described herein for a service or application provided through a Software-as-a-Service platform, in accordance with some embodiments;

FIG. 6 is diagram illustrating the architecture of a model for detecting an anatomical feature of a patient, and that may be used in implementing an embodiment of the disclosure;

FIGS. 7(a)-7(d) are a set of diagrams illustrating an example of a pathology, shown as a binary mask in FIG. 7(a), being expanded using a filter as shown in FIG. 7(b), together with a stochastic operation to randomly expand the pathology pixel-wise as shown in FIG. 7(c) to FIG. 7(d), with these operations being used to simulate a realistic growth of a pathology;

FIGS. 7(e)-7(g) are images illustrating an example of a dental treatment involving a filling and that may be presented to a patient, in accordance with an embodiment of the disclosure;

FIGS. 7(h)-7(m) are images illustrating an example of a progression of bone loss that may occur without proper treatment and that may be presented to a patient, in accordance with an embodiment of the disclosure;

FIGS. 7(n)-7(q) are images illustrating an example of applying background inpainting, in accordance with an embodiment of the disclosure;

FIG. 7(r) are images illustrating a progression of a caries (cavity) that may occur without proper treatment and that may be presented to a patient, in accordance with an embodiment of the disclosure;

FIG. 7(s) is a set of images illustrating a progression of a dental pathology that may occur without proper treatment and that may be presented to a patient, in accordance with an embodiment of the disclosure;

FIGS. 7(t)-7(u) are images illustrating a timeline or progression of periapical radiolucencies (PR) that may occur without proper treatment and that may be presented to a patient, in accordance with an embodiment of the disclosure; and

FIGS. 7(v)-7(cc) are images illustrating a root canal crown procedure that may be presented to a patient, in accordance with an embodiment of the disclosure.

Note that the same numbers are used throughout the disclosure and figures to reference like components and features.

### DETAILED DESCRIPTION

One or more embodiments of the disclosed subject matter are described herein with specificity to meet statutory requirements, but this description does not limit the scope of the claims. The claimed subject matter may be embodied in other ways, may include different elements or steps, and may be used in conjunction with other existing or later developed technologies. The description should not be interpreted as implying any required order or arrangement among or between various steps or elements except when the order of individual steps or arrangement of elements is explicitly noted as being required.

Embodiments of the disclosed subject matter are described more fully herein with reference to the accompanying drawings, which show by way of illustration, example embodiments by which the disclosed systems, apparatuses, and methods may be practiced. However, the disclosure may be embodied in different forms and should not be construed as limited to the embodiments set forth herein; rather, these embodiments are provided so that this disclosure will satisfy the statutory requirements and convey the scope of the disclosure to those skilled in the art.

Among other forms, the subject matter of the disclosure may be embodied in whole or in part as a system, as one or more methods, or as one or more devices. Embodiments may take the form of a hardware implemented embodiment, a software implemented embodiment, or an embodiment combining software and hardware aspects. For example, in some embodiments, one or more of the operations, functions, processes, or methods disclosed and/or described herein may be implemented by a suitable processing element or elements (such as a processor, microprocessor, co-processor, CPU, GPU, TPU, QPU, state machine, or controller, as non-limiting examples) that are part of a client device, server, network element, remote platform (such as a SaaS platform), an “in the cloud” service, or other form of computing or data processing system, device, or platform.

The processing element or elements may be programmed with a set of executable instructions (e.g., software instructions), where the instructions may be stored on (or in) one or more suitable non-transitory data storage elements. In some embodiments, the set of instructions may be conveyed to a user over a network (e.g., the Internet) through a transfer of instructions or an application that executes a set of instructions.

In some embodiments, the systems and methods disclosed herein may be implemented as a set of services or functionality provided through a SaaS or multi-tenant platform. The platform provides access to multiple entities, each with a separate account and associated data storage. Each account may correspond to a dentist, a hygienist, a dental assistant, an insurance company, an analytics company, a dental network, a group of dentists, or an organization, for example. Each account may access one or more services, a set of which are instantiated in their account, and which implement one or more of the methods or functions disclosed and/or described herein.

In one embodiment, the disclosed and/or described image processing technique(s) may be implemented as a backend service on a SaaS platform that provides other services to accounts residing on the platform. In such an example implementation, the operator of the SaaS platform or other form of system may implement the disclosed image processing technique(s) and/or trained models while providing other services or access to other applications for accounts on the platform.

Note that an embodiment of the disclosed methods may be implemented in the form of an application, a sub-routine that is part of a larger application, a “plug-in”, an extension to the functionality of a data processing system or platform, or other suitable form. The following detailed description is therefore not to be taken in a limiting sense.

To address the communication gap between a dentist and patient, dentists utilize diverse tools to inform patients about their dental conditions. One common approach involves using pen and paper to illustrate the problem to help the patient understand. For example, a dentist might draw a tooth's anatomy and depict how issues such as caries (cavities) impact it. Additionally, dentists may employ generic pictures, videos, or animations to facilitate patient understanding. For instance, they may utilize visual aids to demonstrate the progression of caries, showcasing how one can start from dentin then spread into the enamel and ultimately reach the tooth pulp if left untreated, causing an infection at the root. This multifaceted approach helps patients gain a better comprehension of their dental situation and possible concerns.

Incorporation of video as part of providing oral health education can also be an effective tool in improving oral health knowledge, which can impact the oral health behavior of people and communities (see Shah, et al., 2016, Effectiveness of an educational video in improving oral health knowledge in a hospital setting. Indian J Dent. April-June; 7 (2): 70-75). In one study, patients agreed that the visual aids helped them and should be used for all treatment needs identified in a dental office. What seemed to help were images of what the actual disease looked like, which made it easier to identify problem areas in one's own mouth by letting a patient know what to look for (see Momin, et al., 2020, A quality improvement project to assess the use of visual aids to improve understanding and motivation in periodontal patients. BDJ Open. 6:15).

It has been conventional for dentists to use pictures, drawings and/or videos to inform a patient about the status of their oral health and present a treatment plan to the patient. However, dentists struggle to educate patients about the importance of pursuing and completing a treatment to prevent further deterioration of oral health. This is believed to be largely because the tools used to inform patients are generic and patients do not relate them to their own dental situation. Therefore, although showing generic pictures, drawings, or videos are useful, they are not as effective as desired in properly educating a patient and encouraging them to undertake a suggested or recommended treatment.

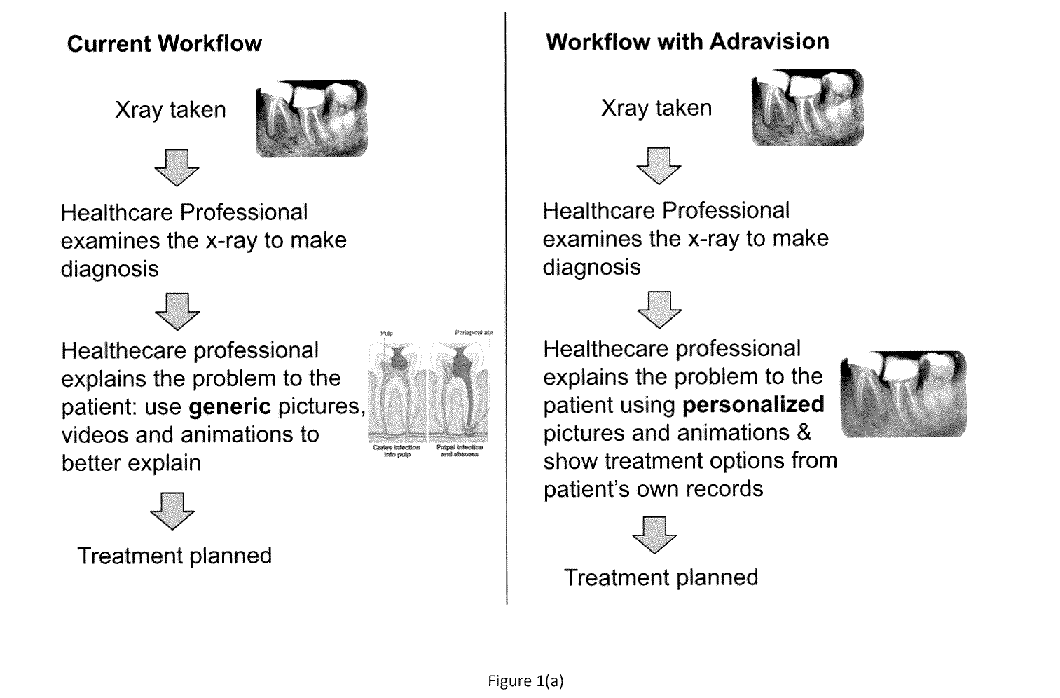

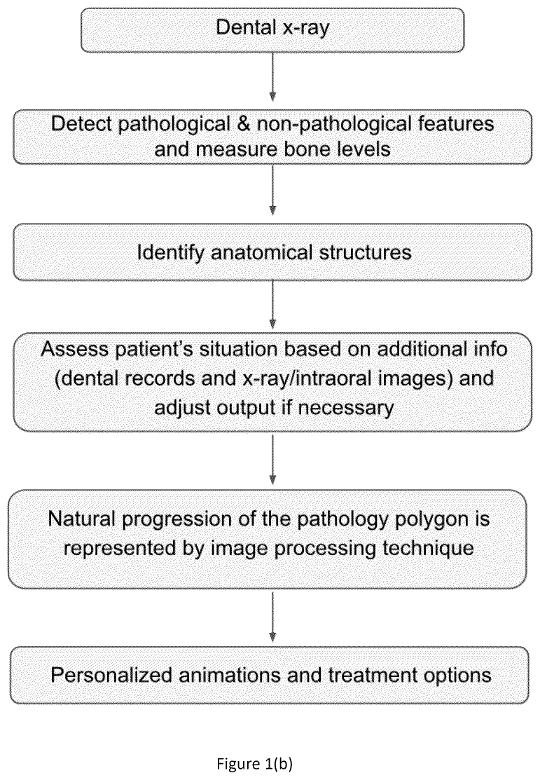

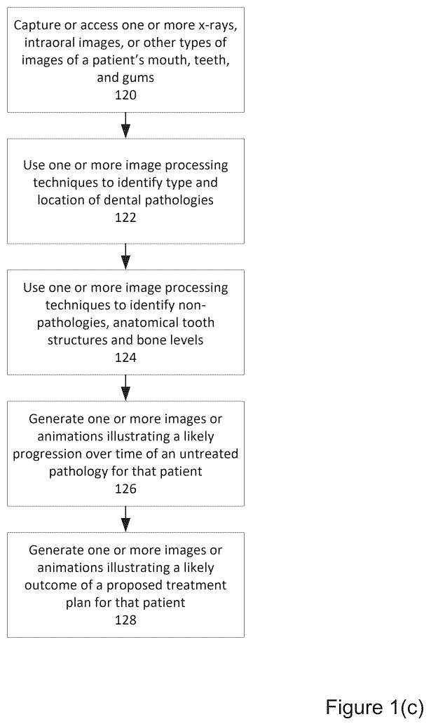

The disclosed and/or described approach (referred to as Adravision or the ADRA platform/system herein) includes multiple capabilities and functions and was developed for the purpose of assisting dental service providers in their practice as well as educating patients. Among other features, it can identify pathologies, non-pathologies (such as previous restorative treatments), dental anatomical structures, and bone levels on dental radiographs, which assists a dentist in planning a course of treatment. FIG. 1(a) is a diagram illustrating an example of the conventional workflow of a dental service provider compared to a corresponding workflow of a dental service provider using an embodiment of the disclosed approach (referred to as Adravision or the ADRA platform/system herein).

The disclosed and/or described approach also serves as a tool to educate patients by leveraging image processing techniques to recognize patterns and anomalies in dental radiographs or intraoral images, which also aids in planning an appropriate treatment for a patient. The tool visually represents the natural progression of a dental pathology (and can become part of a patient's records) and the relationship with the anatomy of a patient's tooth or teeth. By illustrating the progression of a pathology using visual means, an embodiment can display how untreated diseases can impact different anatomical features over time, and thereby emphasize the importance of obtaining timely treatment. Further, because the generated images or animations are specific to an individual patient, they are expected to be more effective at both educating the patient and encouraging them to obtain treatment.

Adravision (whether provided as a client-side application, a service provided through a remote data processing platform, or a combination of those access mechanisms) makes patient education personalized by creating images and/or animations based on a patient's dental records. In this regard, Adravision has the capability to generate personalized animations using a patient's own dental records, x-ray/intraoral scans, and related dental or health information. By utilizing patient specific data, the disclosed approach creates animations that directly illustrate the individual's dental conditions, treatment options, and potential outcomes of treatment or a lack thereof. As a result, the disclosed and/or described approach can present personalized images/animations that not only educate a patient about their dental problem, but also show the progression of a disease if proper treatment is not sought, as well as illustrating the effect of one or more treatment plans.

This personalized approach enables patients to visualize the status of their own dental health, understand the implications of various treatment choices, and better comprehend potential benefits and risks associated with those treatments. Moreover, by demonstrating personalized disease progression, Adravision can help patients to better comprehend the expected trajectory of their dental condition(s), making it easier to grasp the urgency or significance of a proposed treatment.

Such a more personalized approach is expected to significantly enhance patient understanding by providing visual representations that simulate disease progression in their own case and with reference to their own teeth, with a result of motivating patients to consider and proceed with recommended treatments to prevent further deterioration of their dental health. This approach also contributes to building trust between the dentist and the patient, as it allows for a more collaborative and informed decision-making process, ultimately leading to better treatment outcomes and increased patient satisfaction.

Conventionally, there are dental patient education applications available, although these are all lacking in one or more important aspects. Many focus on providing general information about treatments, outcomes, and disease progression without offering personalized content that is more effective in communicating a patient's current condition and treatment options.

Chapter2Dental (as an example) provides videos demonstrating various treatments and their outcomes but lacks the benefit of personalization tailored to an individual patient's specific dental conditions based on a patient's dental radiographs and/or intraoral images. This limits its effectiveness in conveying information and options to the patient. One reason for this is because It prevents a patient being able to “connect” the images to their own teeth and oral conditions.

Overjet.ai or hellopearl.com use colorized polygons to highlight dental pathologies or features. Although this helps dentists better understand an x-ray record and, in some cases, may assist in educating a patient, there is no ability to show the consequence of not pursuing a specific treatment (or the benefit of pursuing a specific treatment plan). This limits the effectiveness of these approaches in demonstrating the positive aspects of a treatment plan to a patient.

The absence of personalization in conventional approaches to educating patients limits their effectiveness in engaging patients and helping them comprehend the relevance of treatments to their unique dental situation. In contrast, it is believed that by customizing educational content based on a patient's own dental pathology, anatomy, and proposed treatment plan, embodiments of the disclosure will enhance patient understanding and their motivation to pursue necessary treatment(s). Providing tailored educational materials and visualizations that illustrate how specific treatments will impact an individual's oral health is also expected to lead to better patient engagement and adherence to treatment recommendations.

Embodiments of the disclosure (i.e., Adravision) personalize each treatment option based on the patient's dental pathology as detected on radiographs and/or intraoral images and generate customized educational content for a patient. This tailored approach ensures that educational content is more relevant to each patient's oral health needs. In one embodiment, by utilizing animation on a patient's own dental radiographs or intraoral images, Adravision offers patients a more accurate and complete overview of their oral health. Such a visualization is expected to assist patients to better understand the complexities of their dental condition(s), and the expected result of pursuing or not pursuing a recommended treatment.

In one embodiment, one or more convolutional neural network machine learning models are used to process images and to (i) identify the type and location of dental pathologies (such as caries, periapical radiolucency, furcal involvement, attrition, and calculus) and non-pathologies (such as filling(s), crown(s), root canal treatment(s), implants and pontics), (ii) measure bone levels from the cementoenamel junction to the bone levels or root, and (iii) identify anatomical tooth structures (such as dentin, enamel, and pulp). These techniques and models are described in greater detail herein with regards to the training data used and the operation of the trained model or models.

After identifying these features, additional image processing techniques are employed to (i) detect the severity of the pathology based on how deep it is inside a tooth (based on its relationship with the identified anatomical features), (ii) illustrate the expected disease progression, showing how untreated conditions may worsen over time, and (iii) illustrate one or more treatment options and how they impact the patient's condition. It is expected that this combination of features will be a powerful tool to emphasize the importance of obtaining both timely and the correct type of treatment.

Embodiments combine personalized treatment planning, customized educational content, a more comprehensive oral health visualization obtained through images and/or animation, and a realistic simulation of the progression of a dental disease or problem. This provides a comprehensive solution aimed at improving patient understanding and engagement in their dental care. The personalized approach is expected to help motivate patients to take the necessary steps for their oral health based on a more complete understanding of their specific conditions and treatment options.

In one embodiment, when a dental radiograph is received by the Adravision software at a remote platform (typically from a dental office using a dedicated application), the image is processed through one or more machine learning model services. The image is stored “in the cloud” (i.e., on the platform) and the inference results are stored in an associated database. The inference results are then provided to the processing scripts (which execute one or more functions) to generate personalized patient animations illustrating the impact of treatment or a lack of treatment.

The generated animations and any accompanying images or information are then made available to a dental services provider (again through the Adravision application or workstation) for presentation to the patient as part of a discussion of the patient's dental situation and treatment options.

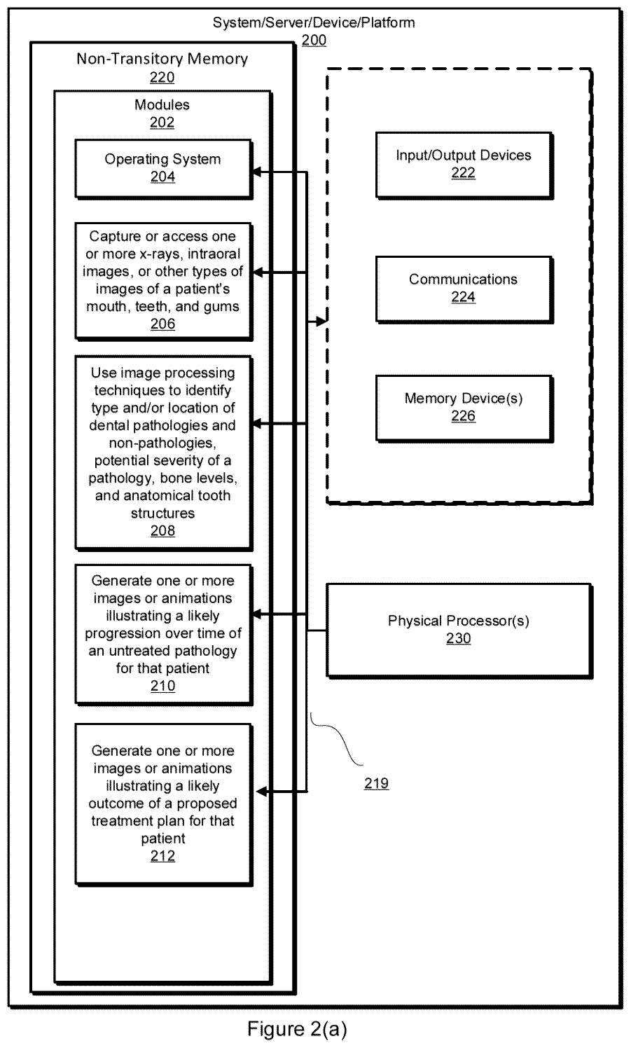

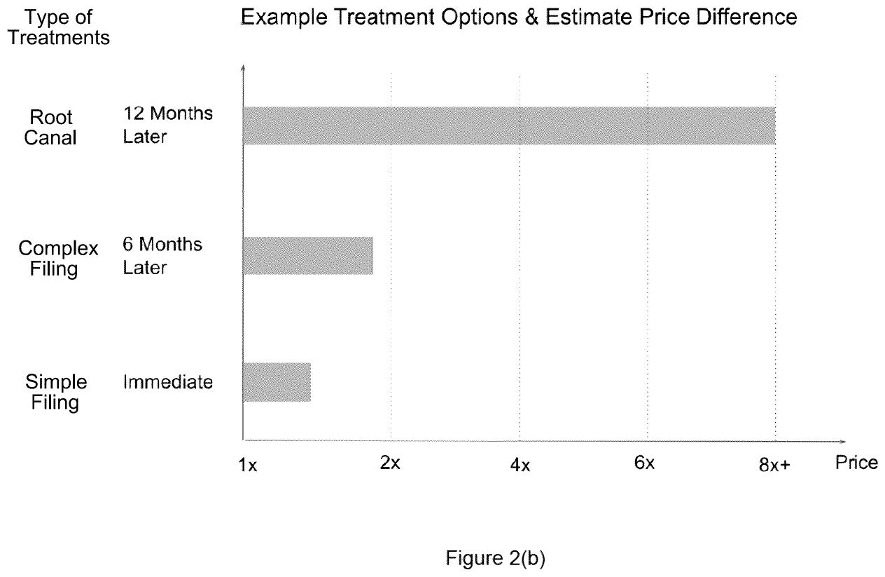

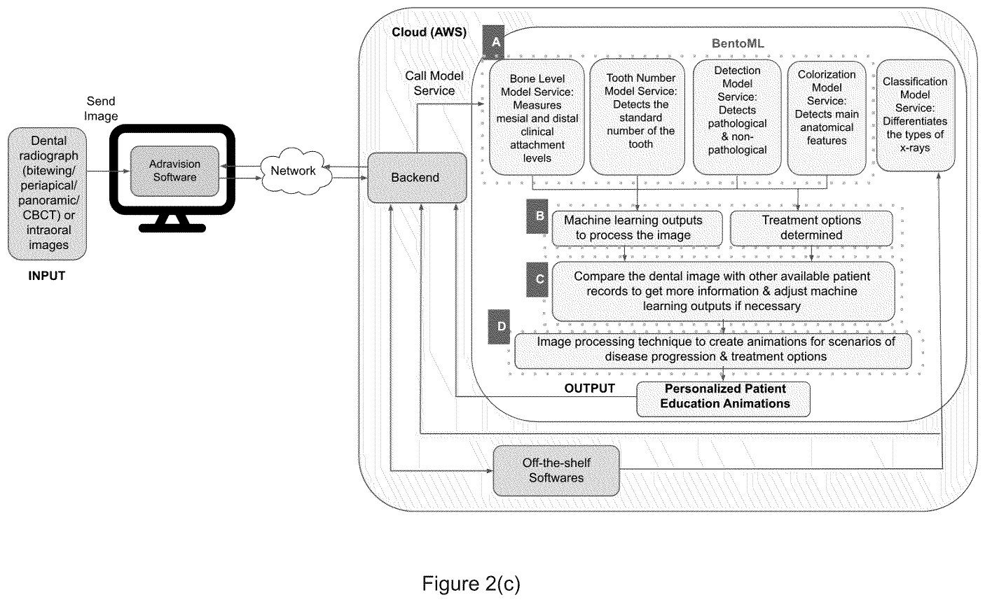

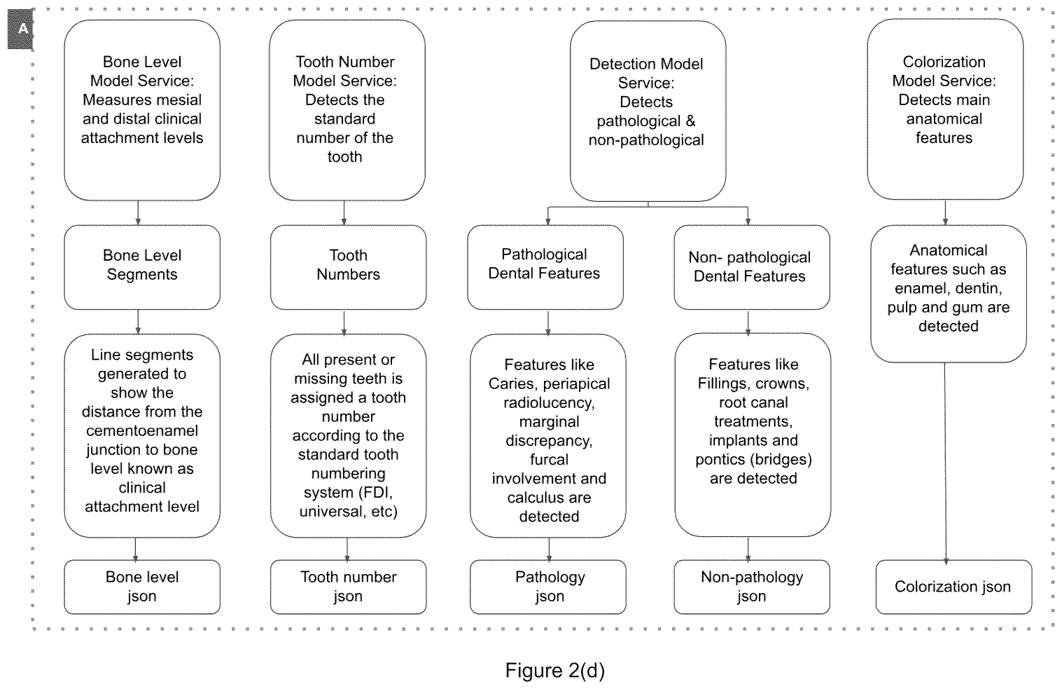

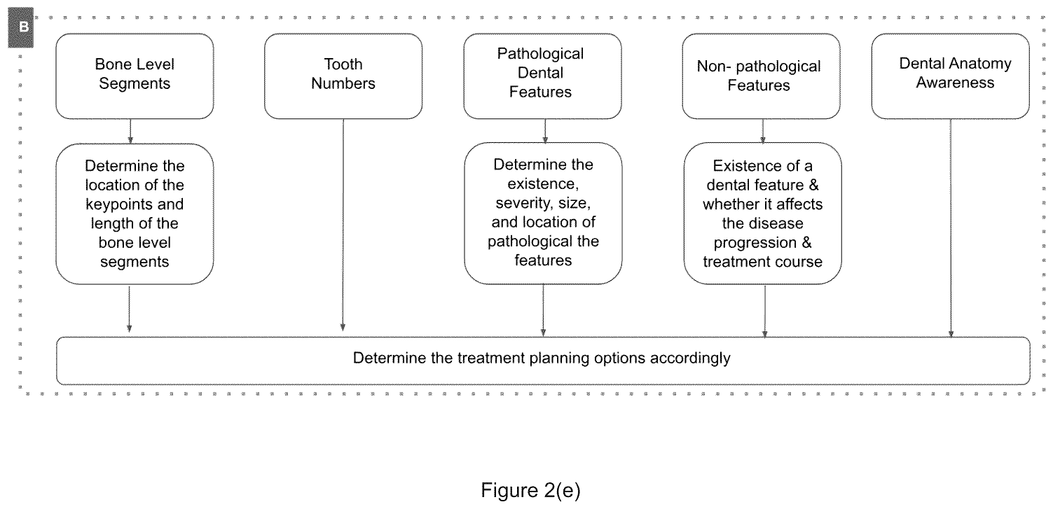

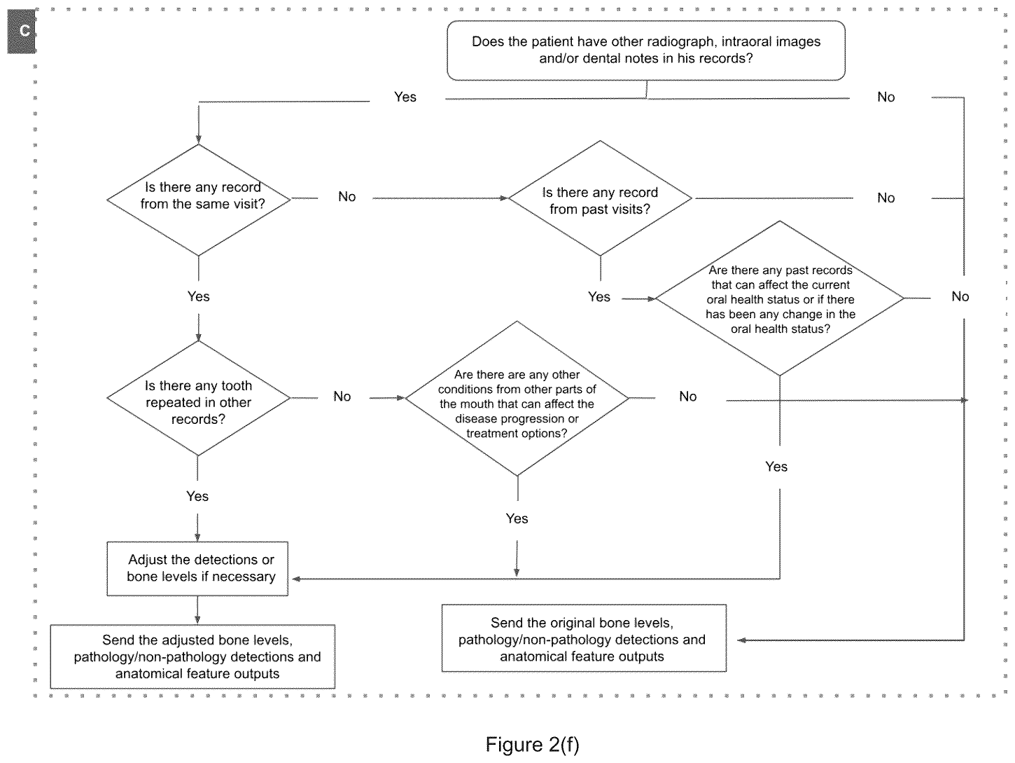

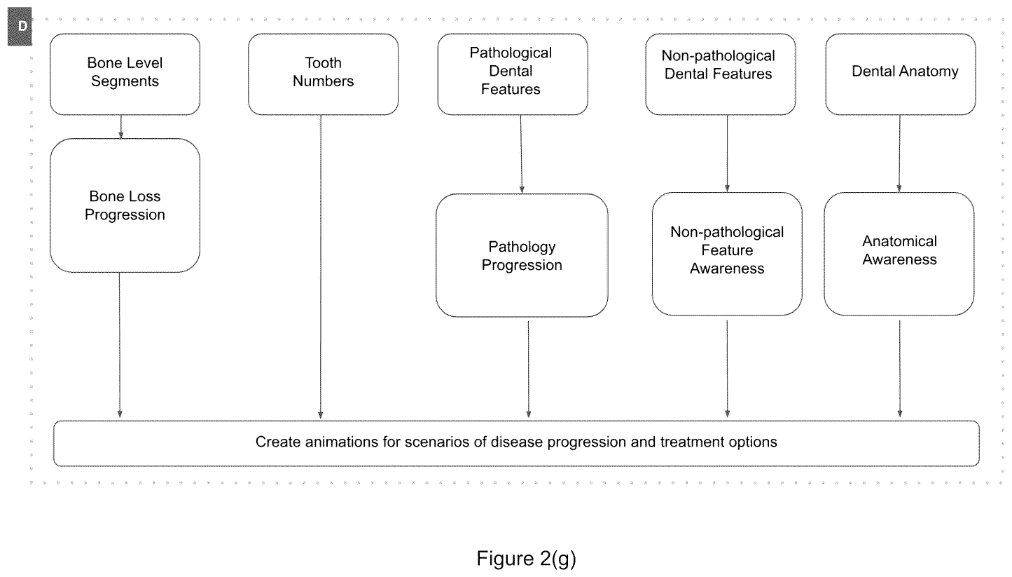

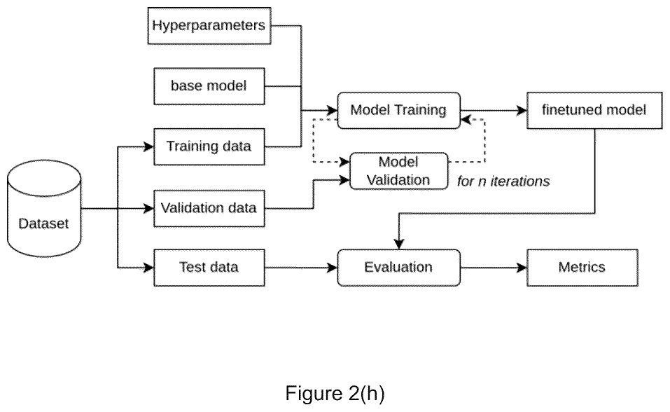

The indicated figures provide additional details regarding the processing steps or stages. FIG. 2(c) is a diagram illustrating the elements, components, services, and processes in an example embodiment of the disclosed and/or described system and architecture. As shown in the Figure, in one example embodiment and use case, an image or images obtained from a patient's mouth (indicated as a “Dental radiograph” or “intraoral images” in the figure) are provided or captured by an application (illustrated as “Adravision Software”) in a client device or workstation. The radiographs may include one or more of bitewing, periapical, panoramic, or CBCT images as non-limiting examples. Typically, the client device would be located in a dental office and may be interconnected with a camera, x-ray machine, or other suitable image generating device or process.

The image or images are provided over a network to the server platform (illustrated as Cloud (AWS), as an example), where the images are processed by one or more models to generate information and data used to create the animations or images that will be shown to the patient. As suggested by the Figure, these models may be provided as “services” and may include one or more of (as indicated by “A” in the figure and shown in greater detail in FIG. 2(d)):
Bone level model—measures mesial and distal clinical attachment levels;
Tooth number model—identifies teeth and determines standard number or identifier for each tooth;
Detection model—operates to detect pathological and/or non-pathological features in the patient's mouth;
Colorization model—detects and indicates the primary or main anatomical features of each tooth and its surrounding;
Classification model—differentiates between radiograph or intraoral image, as well as the specific type of X-ray (such as periapical, bitewing, panoramic, or CBCT).

The output or outputs of each model are then used to process the image or images, and to determine one or more possible treatment options (as illustrated by “Machine learning outputs to process the image” and “treatment options determined” in the figure and indicated by “B” in the figure and shown in greater detail in FIG. 2(e)). For example, for smaller detected caries, a simple filling may be recommended, whereas for larger caries that have reached the pulp and infected the root, a root canal followed by a crown may be suggested.

The processed image or images and treatment option(s) are combined and/or interpreted in view of the patient's dental and medical records (where available) to generate a more complete understanding of the patient's dental and/or medical condition (as indicated by “C” in the figure and shown in greater detail in FIG. 2(f)). A comparison may be used to investigate whether changes should be made to the output from the detection or measurement models, particularly in relation to factors that may affect disease progression or determining a desirable treatment plan. As an example, the patient records of prior dental and/or medical conditions may provide information that impacts a selected treatment, such as allergies, previous dental problems that have worsened, or patient behaviors that impact the teeth or jaw.

The processed image or images are then used to create one or more animations for viewing by the patient in coordination with the dentist or dental services provider (as indicated by “D” in the figure and shown in greater detail in FIG. 2(g)). These are indicated by “personalized patient education animations” in the figure. As disclosed herein, these animations may provide guidance on one or both of the patient's present dental condition and how it may worsen if left untreated (addressing both clinical and financial implications), and on how a suggested treatment would progress and provide an improvement.

FIG. 2(b) is a chart for illustrating to a patient various treatment options and the relative cost, for their consideration when deciding upon a treatment plan in coordination with a dentist. The chart also highlights how delaying treatment can lead to increased financial costs over time.

FIG. 2(d) is a diagram illustrating a set of services or processes that may be implemented by a model or models that are made available through the system architecture of FIG. 2(c) in an example embodiment of the disclosed and/or described system and architecture. As shown in the figure, each of the models referred to with reference to FIG. 2(c) operate to determine one or more of the indicated data or information:
Bone level model—generates line segments that indicate the distance between a cementoenamel junction to a bone level (referred to as the clinical attachment level);
Tooth number model—assigns a number to present or missing teeth using a standard numbering system or identifiers;
Detection model—identifies pathological features, including caries, periapical radiolucency, calculus, furcation and attrition among other possible forms, and identifies non-pathological features including fillings, crowns, root canal treatment, implants, pontics (e.g., bridges), wisdom tooth removal, and inferior alveolar nerve treatment, among possible forms;
Colorization model—used to detect and identify anatomical features such as enamel, dentin, and pulp;
Classification model—detects and identifies type of image, such as a radiograph or intraoral image, as well as the specific type of X-ray (such as periapical, bitewing, panoramic, or CBCT).
   In one embodiment, each of the model or service outputs is represented as a JSON, which is a standard format for data storage.

FIG. 2(e) is a diagram illustrating a set of services or processes that may be implemented to determine a desirable treatment plan in an example embodiment of the disclosed and/or described system and architecture. As shown or suggested by the figure, determining a treatment option or options may involve data and information produced by one or more of the example models/services disclosed. One or more of the sources of data or information may be subject to a threshold operation or severity evaluation (typically performed by a model and/or dental services provider) as part of determining a recommended treatment or an option for treatment.

As non-limiting examples, a severity evaluation may consider a tooth and its condition, a dental feature and its location relative to a specific tooth, a size or dimension of a dental feature, and an evaluation or judgment as to whether a dental feature may impact a proposed treatment. In one example, a severity or impact evaluation may be performed using a trained model. In another example, such an evaluation may be performed by reference to pre-determined values set by a filter in response to a dentist's inputs.

FIG. 2(f) is a flow chart or flow diagram illustrating a set of services or processes that may be implemented to compare one or more dental images with other available patient records. This may be done to obtain additional information regarding the patient's dental and/or medical history as part of determining a desirable treatment plan.

In the process flow illustrated, a service provider may search for and access other medical or dental records of a patient (such as other dental radiographs, intraoral images, or dentist notes, as non-limiting examples) to identify prior or contemporaneous medical or dental conditions or treatments that might impact the recommended treatment or efficacy of a treatment. If so, corrected values for one or more of bone levels, pathological or non-pathological features, or anatomical features may be generated and utilized to correct or otherwise modify a model output.

For example, if bone levels are initially measured using a periapical radiograph, the patient's records may be queried to determine if a bitewing radiograph for that area of the mouth is available. In such cases, the bone levels on the bitewing radiograph may be utilized instead, as the bitewing radiographs usually provide a better assessment of bone health. In another example, if a caries is detected in a panoramic radiograph, the patient's records may be queried to determine if a bitewing or periapical radiograph for that area of the mouth is available. If no caries is visible in the bitewing or periapical radiograph, no treatment will be pursued, as bitewing and periapical radiographs are considered more accurate for caries detection than panoramic radiographs.

FIG. 2(g) is a diagram illustrating a set of services or processes that may be implemented to generate an animation of a scenario of disease progression and treatment options in an example embodiment of the disclosed and/or described system and architecture. As shown or suggested by the figure, the data and information derived from the models or otherwise related to the indicated categories (bone level segments, tooth numbers, pathological dental features, non-pathological dental features, and dental anatomy, as examples) may be processed and then provided to a service that operates to generate one or more animations illustrating disease progression in the absence of treatment and/or disease progression when treated. If more than a single treatment option is being considered, then an animation illustrating disease progression when treated may be generated for each such option.

In one embodiment, the disclosed service or services on the platform use image processing techniques to visually represent the natural progression of a pathology polygon, illustrating how the disease can impact anatomical features.

For illustrating a Caries Progression, a combination of “dilation” and stochastic expansion operations are used to expand the cavity area in a realistic looking way, to illustrate what would happen if a cavity were left untreated and progressed naturally. In one example, this uses a 7×7 disk kernel element with an anchor point in the center of the kernel to dilate the cavity area. The function dilates the cavity mask (a binary mask of the same size as the image, where the pixels belonging to the cavity have a value of 1 and the rest have a value of 0) by a certain number of pixels in all directions. Stochastic Expansion may then be used to add noise to the straight and smooth edges produced by dilation. In stochastic expansion, pixels of the edge of the cavity are randomly set to 1 resulting in a more irregular shape of outline dilation.

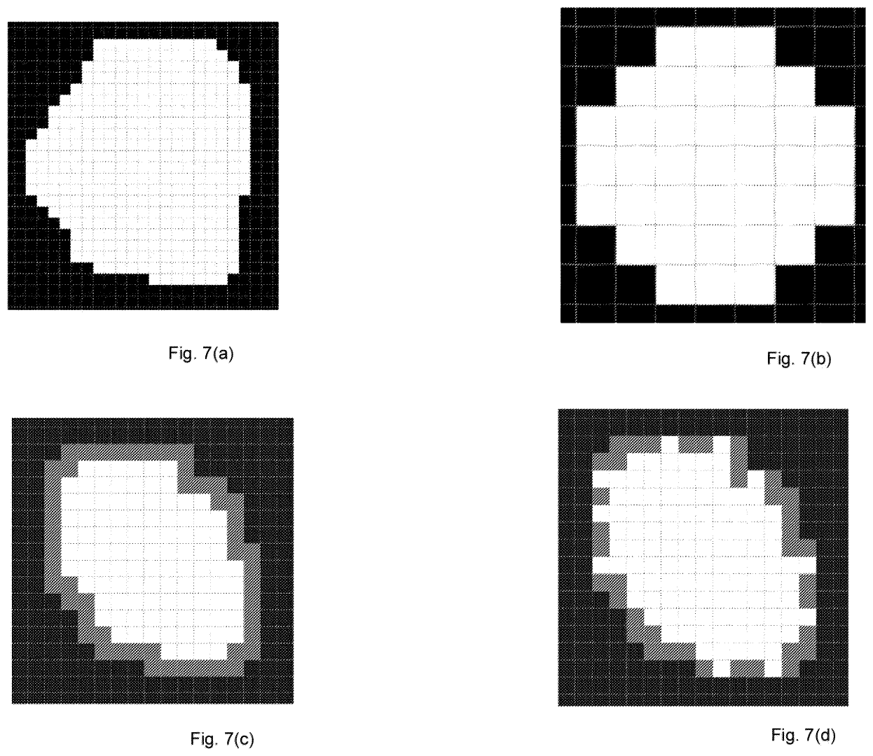

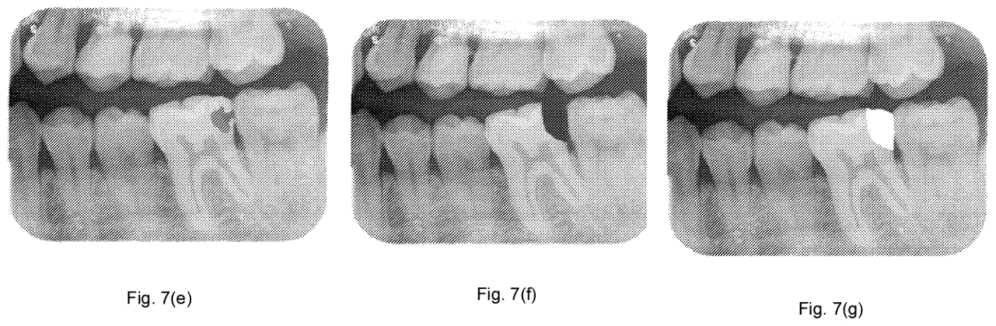

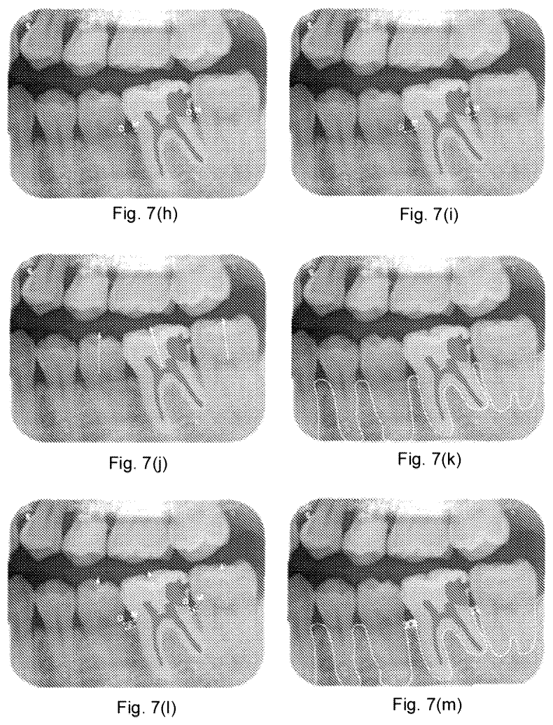

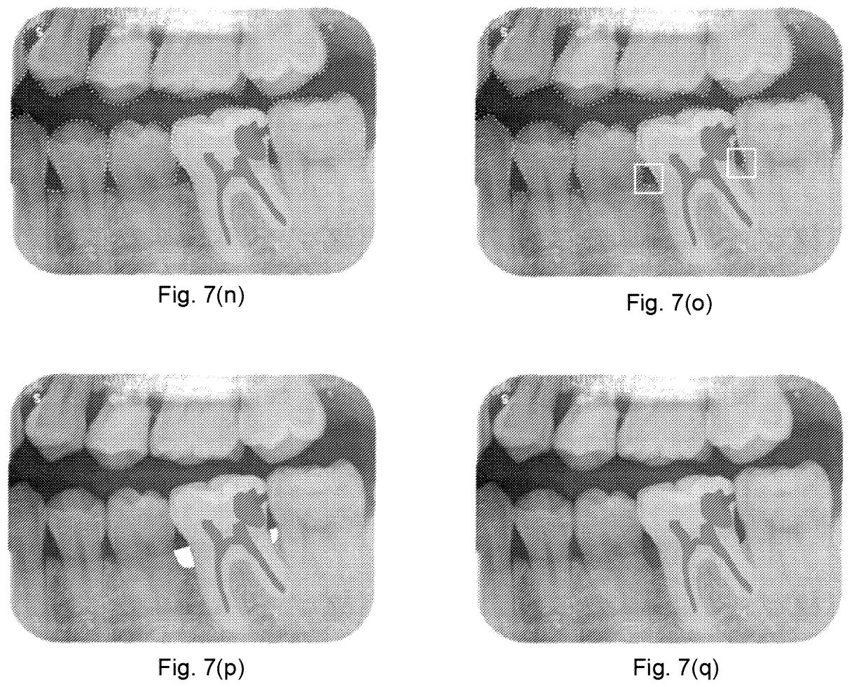

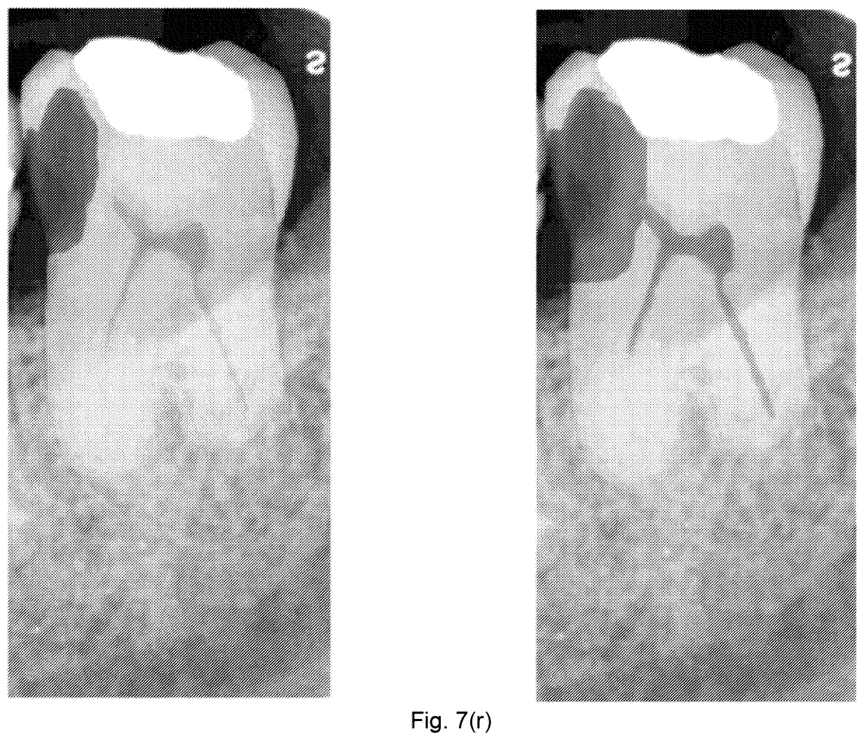

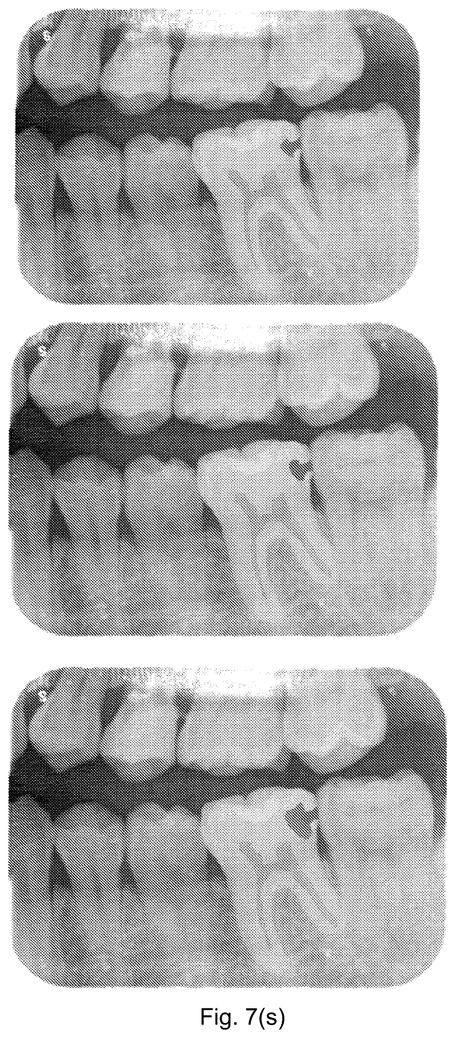

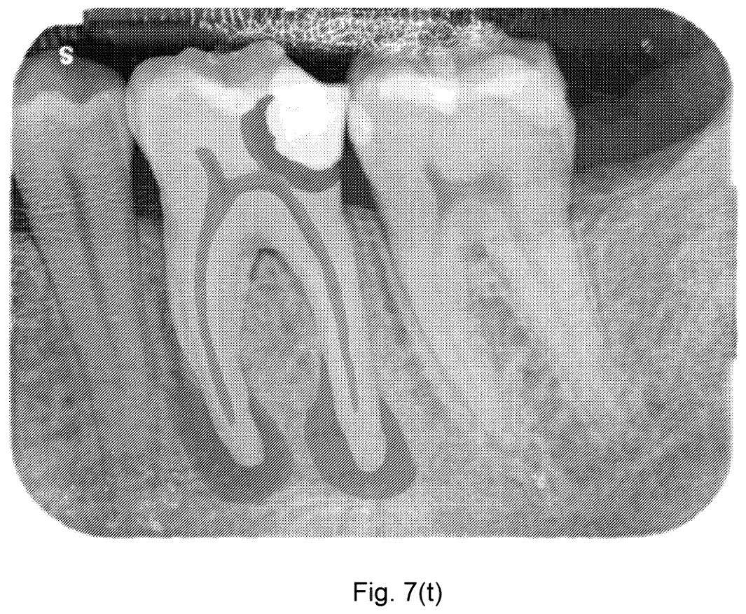

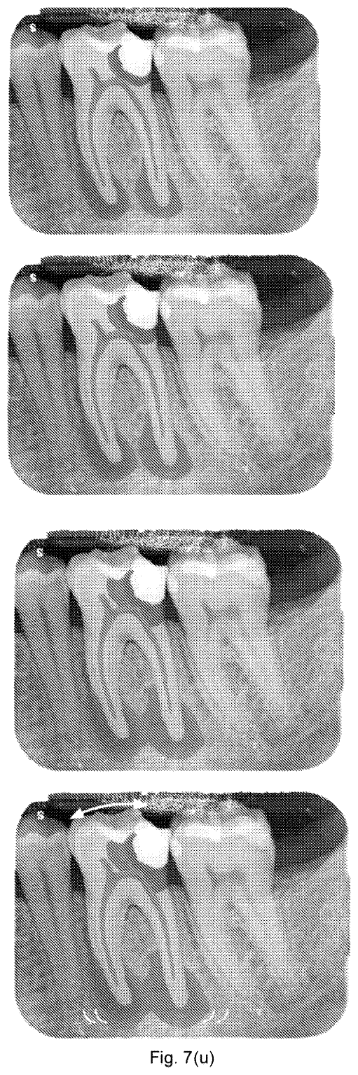

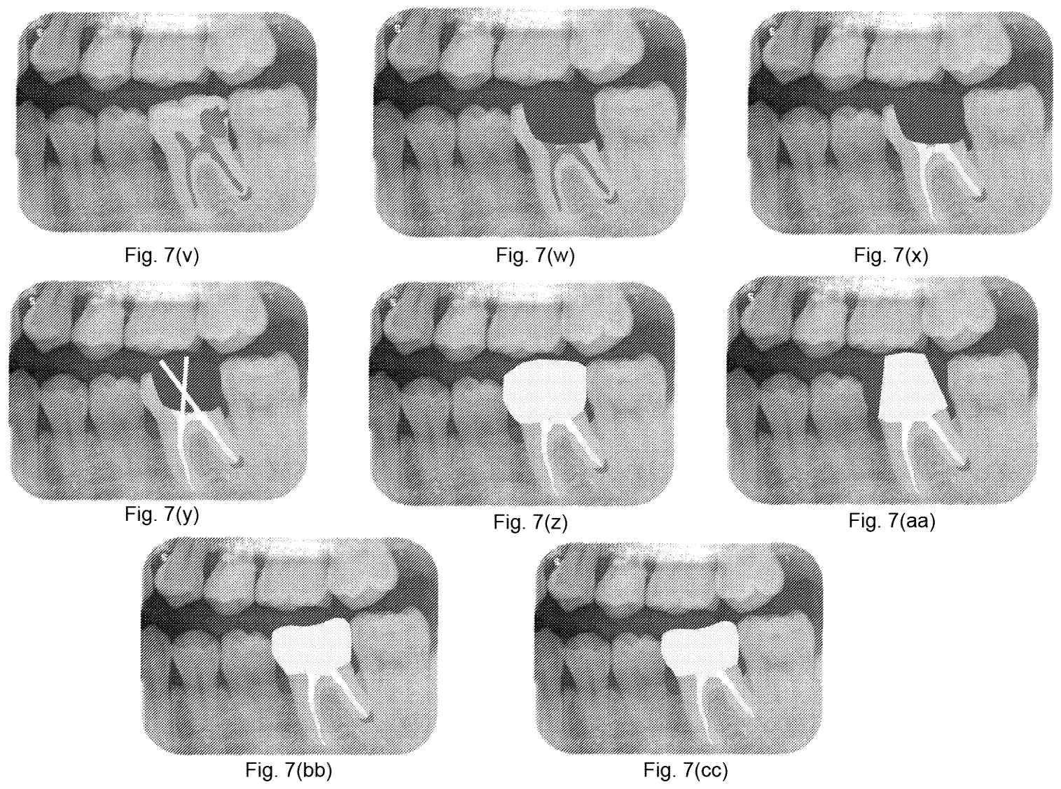

FIGS. 7(a)-7(d) are diagrams illustrating how the dilation and stochastic expansion operations function in an embodiment of the disclosure. A pathology is shown as a binary mask in FIG. 7(a), being expanded using a filter as shown in FIG. 7(b), together with a stochastic operation to randomly expand the pathology pixel-wise as shown in FIG. 7(c) to FIG. 7(d). Together these operations are used to simulate a realistic growth of a pathology.

For anatomical and non-pathological awareness, the expansion rate of the cavity is different in the anatomical features, just as in reality. A cavity will expand at a much faster rate in the dentin than in the enamel, while a cavity does not expand in fillings, crowns, and bridges. The rate of expansion is determined by the number of iterations in which the dilation operation is performed, i.e., for every iteration of dilation of the cavity in the enamel. In one embodiment, five iterations are performed for the dilation of the cavity in the dentin. When the expansion of the cavity reaches the pulp of the tooth, the pulp is colored brown (or changed in color) to indicate an infection of the pulp. An implementation in the Python version of OpenCV may be used to perform this operation.

For Periapical Radiolucency and tooth instability, instances of periapical radiolucencies may be indicated by dark(er) areas around the roots of a tooth. They are expanded using the same dilation operation as used for caries, and only expand in bones and not to other teeth. An example of periapical radiolucency at the tips of the roots of a molar is shown in FIG. 7(t). When the area of the periapical radiolucency exceeds a certain threshold, the tooth may become unstable due to the lack of bone structure to support it. In this situation, an outline of the tooth is used to crop and animate the tooth rocking back and forth, by indicating a white double arrow above it to assist the dentist and patient (as illustrated in FIG. 7(u)).

Bone Loss Progression is indicated in one embodiment by a drop in bone level, which is represented in an x-ray. An arrow may be used to indicate the direction of the bone level as bone loss progresses. The orientations of the teeth, which are determined by a model, can be used to determine the direction of the recession.

Another model may be used to locate the bone level points. These points may be indicated by the ends of the line segments in an image or animation. Without a progression, the bone level may initially be represented by connecting the bone level key points and forming color coded line segments. As bone loss progresses, the line segments are translated downwards, while tracking the contours of the teeth which are the output of a tooth segmentation model.

The personalized and patient specific animations or images generated using an embodiment of the disclosure may be used to illustrate the progression of a pathology polygon, illustrating how a disease can impact anatomical structures. A personalized animation can be used to illustrate the progression of bone levels, illustrating how bone loss can impact anatomical structures leading to a “shaky” tooth. Similarly, personalized animation may be generated to illustrate how an immediate treatment of the caries with a filling will stop the progression of the caries. Likewise, a personalized animation may be generated to illustrate how late or delayed intervention may result in a root canal and a crown treatment.

With regards to the generated animations, the following provides additional guidance and implementation details to configure and operate the disclosed and/or described models and processing pipelines:
Generating Pathology Progression Animation
   Principles
   The techniques used to generate the animations should make them look organic and realistic. This may be achieved by the overall smooth growth of the pathology (caries, periapical radiolucency, furcal involvement, as examples) with some randomness, similar to the gradual creeping of actual pathologies as they grow.
   The progression pathology should also be aware of the anatomy, e.g., a cavity grows slowly in a tooth's enamel, but very quickly in its dentin. The animation is generated so that the relative growth speed is accurate in different tooth parts;
   Mechanism
   The expansion of pathologies (caries, periapical radiolucency, furcal involvement as example) begins with a binary raster mask with a blob of white pixels (i.e., a value of 1) indicating the area of the pathology and black pixels (a value of 0) indicating the background;
   The shape is expanded in expansion cycles. An expansion cycle is a combination of (1) Dilation to grow the mass of the blob, and (2) Stochastic expansion of border pixels for non-uniform edges. As mentioned, a pathology is shown as a binary mask in FIG. 7(a), being expanded using a filter as shown in FIG. 7(b), together with a stochastic operation to randomly expand the pathology pixel-wise as shown in FIG. 7(c) to FIG. 7(d). Together these operations are used to simulate a realistic growth of a pathology;
      Dilation is executed by a convolution operation performed by a disk kernel with an anchor point in the center of the kernel (for example a 7×7 disk kernel). A single dilation operation expands the blob by approximately 3 pixels in all directions;
      Stochastic Expansion—to add noise to the straight and smooth edges produced by the dilation, pixels from a 1-pixel thick outline around the blob are randomly chosen to set to 1. The probability is set to 0.2 by default;
      Anatomy Awareness
      Cavities only grow in the enamel, dentin, and pulp. Periapical involvements only expand in the bone. To prevent pathologies from growing in all directions (e.g., into space or the bone), instance segmentation masks of tooth anatomy are used.
      Pathologies will only expand in regions that overlap with the relevant segmentation masks and at speeds that reflect the relative speed(s) of growth. For example, cavities expand in enamel very slowly relative to their expansion in dentin.
      The number of expansion cycles per animation frame controls the expansion speed in different regions. In one embodiment, the expansion cycle is done once every 7 frames for cavity expansions in enamel and every frame in dentin so that the cavity expands faster in dentin.
Generating Bone Loss Progression Animation
   Extending Bone Loss—the bone loss progression animation is more reliant on anatomical context. In one embodiment, the inputs required, as shown in FIG. 7(c) are:
   Bone level points of the mesial and distal surface of every tooth as shown in FIG. 7(h), detected by a key point detector deep learning model;
   Connection between adjacent surfaces by connecting the bone level keypoints, based on the positions of the teeth and their gaps, as shown in FIG. 7(i);
   The vertical axes of teeth as shown in FIG. 7(j), measured by a tooth orientation model;
   Segmentation mask of bone structure shown as a solid white line in FIG. 7(k), as detected by a semantic segmentation model;
Using the above inputs, the following values are derived:
   Direction of bone loss—this is determined based on the vertical axes of the teeth. The bone loss will happen in the opposite direction, as shown by white arrows in FIG. 7(l);
   Simulated bone loss areas as shown in FIG. 7(m)—these areas are cut off when the bone level line is moved down in the direction opposite to the vertical axes of the teeth;
Background Inpainting-a final (or nearly final) step requires that the areas of simulated bone loss are replaced with the background. Background refers to the dark empty spaces that are neither teeth nor bone;
   In one embodiment, this is achieved using the following:
      Get the segmentation mask of the background of the X-ray, shown as a dashed line in FIG. 7(n);
      Get the median shade of gray from pixels in areas of the background that is close to the bone level points, shown as white squares in FIG. 7(o);
      Paint/color the areas of simulated bone loss, shown as white areas in FIG. 7(p) with this shade of gray;
      Blend the areas into the original image at 85% opacity to look more realistic, as shown in FIG. 7(q).
After (in)painting, the areas of simulated bone loss look like missing bone.

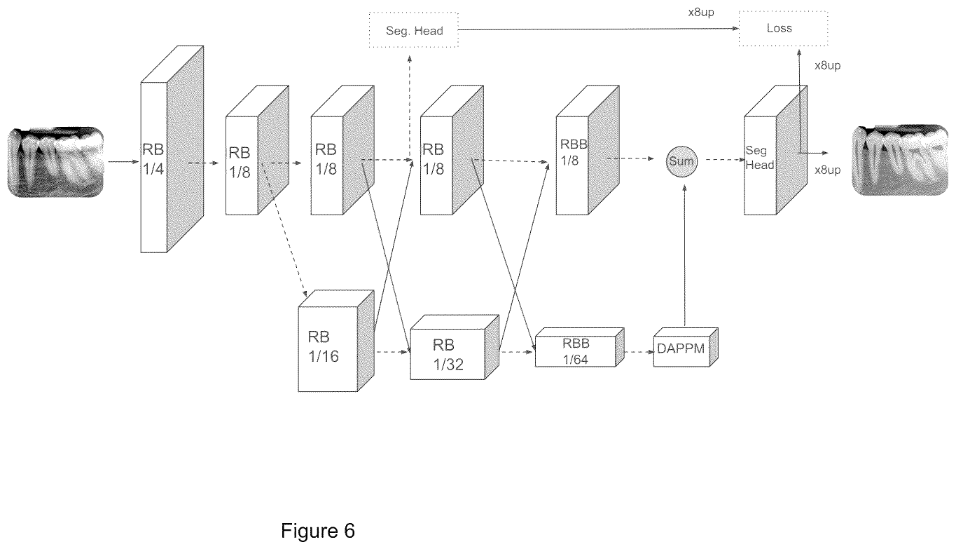

As disclosed and/or described, embodiments may incorporate trained models that operate to identify or otherwise determine one or more features found in a patient's images (such as by operating as a classifier). Each model requires training, and an example of a training process is provided in the following:
Pathology and Non-pathology Detection Machine Learning (ML) Model Description and Training
   The disclosed system includes a local software application and backend (remote server or platform resident) services that utilize computer vision (e.g., image processing) and machine learning to identify caries and periapical radiolucency in dental radiographs (as non-limiting examples);
   To train the model, thousands of dental radiographs were obtained from diverse dental clinics around the world. The dataset included 2D dental radiographs namely, bitewing, periapical and panoramic radiographs;
      Each radiograph was labeled for pathologies (i.e., caries, periapical radiolucency, calculus, as non-limiting examples), and non-pathologies (i.e., fillings, crowns, root canal treatments, implants, pontics, and wisdom tooth removal, as non-limiting examples) by experienced dentists using an annotation platform. The labels were then reviewed by another dentist, and adjustments were made to improve the accuracy of the labeling, such as by adjusting the size of a label, adding or deleting an annotation, or changing the classification of the label;
      The labels underwent preprocessing steps such as resizing and/or padding before being fed through a YOLOv5 instance Segmentation model to detect and outline the pathologies and non-pathologies. Model customizations may be achieved by optimizing settings, augmentations, and hyperparameters;
      FIG. 2(h) shows the workflow of a training process for detection and identification of pathological and non-pathological features. Post processing steps such as filtering bounding box predictions using two thresholds-Intersection over Union (IoU) threshold and confidence threshold-were used to remove overlapping duplicated detections of the same feature and low confidence detections. This removes false positives for a more optimal balance between precision and recall;
Bone level Measurement ML Model Description and Training
   To train the bone level measurement model, thousands of dental radiographs were obtained from dental clinics around the world. The dataset included 2D dental radiographs namely, bitewing, periapical and panoramic radiographs. Each radiograph within the dataset had the individual teeth cropped out and the labelers were given individual tooth crops to label. In some cases, a tooth crop may have included multiple teeth, in such cases the labelers were asked to annotate the tooth in focus;
      The dataset was labeled for the clinical attachment level (CAL) measurement which is the distance from the cementoenamel junction (CEJ) to the level of the bone (BL). Each radiograph was labeled by a dentist or dental nurse/assistant on a labeling platform or by trained professionals on a different platform. Each line segment was reviewed by a dentist and any necessary adjustments were made;
      The input image data underwent preprocessing. For example, as part of preprocessing, the images were resized to a specific size and the keypoints were translated accordingly. The images were fed into a YOLO pose estimation model, as implemented by Ultralytics. The model was trained using the extra-large (x) variant and used version 8 of the YOLO model architecture;
      The model was trained from scratch-without starting from the provided pretrained weights. The pose estimation model from YOLO detects keypoints. The model is fed not only the keypoints for BL and CEJ but also a small bounding box of a fixed size for each keypoint. The confidence score of the bounding boxes encodes the visibility score, which will be used to identify keypoints that are not visible due to occlusion or x-ray artifacts;
      During post processing, the outputs (keypoints class and coordinates) are matched with the bounding box in the same location in order to get the visibility of the keypoints. The overall training workflow was similar to the one described for the Pathology and Non-pathology Detection;
Anatomy Detection ML Model Description
   A semantic segmentation model was trained to classify pixels as belonging to one of the following categories: enamel, dentin, pulp, or background/others. The training dataset consists of polygons of each category drawn manually by labelers. The polygons are then converted into masks and combined into a pixel-level classification mask. A state-of-the-art model DDR-Net was used as the segmentation model. It was found to achieve the best trade-off between accuracy and inference speed. An example architecture for this model is shown in FIG. 6;
      In the figure, “RB” denotes sequential residual basic blocks. “RBB” denotes the single residual bottleneck block. “DAPPM” denotes the Deep Aggregation Pyramid Pooling Module. “Seg. Head” denotes the segmentation head. Black solid lines denote information paths with data processing (including up sampling and down sampling) and black dashed lines denote information paths without data processing. “Sum” denotes the pointwise summation. Dashed boxes denote the components discarded in the inference stage. The Figure and caption were reproduced from that found at: https://arxiv.org/pdf/2101.06085.pdf.

In one example embodiment, a radiograph in the form of bitewing, periapical, or a panoramic image is received by the disclosed application (the Adravision client, system or platform). Using the dental office's network, the local software application sends the image to the backend platform (as suggested by FIG. 2(c)). The backend/platform calls one or more model services to:
Classify the type of image;
Use the Bone Level Model Service to measure bone level segments;
Use the Tooth Number Model Service to identify a tooth number associated with each tooth;
Use an Instance Segmentation Model Service to detect and outline pathological and/or non-pathology features; and
Use a Semantic Segmentation Model Service to colorize anatomical features.

From the bone level segment measurements, the detection of pathological and/or non-pathological features (previous restorative treatments, as an example), the colorization of dental features, and the identification of associated tooth numbers, the process generates polygons/masks which are sent to the backend server or platform (if not generated at that location). Image processing techniques are then used to visually depict the progression of identified pathologies over time or in stages. This feature helps to illustrate how diseases or issues can impact a patient's dental anatomy.

The acquired data and information, and generated images or animations are provided by the platform to the local application for access by a dental professional as part of presenting a personalized education to a patient. In one embodiment, the backend of the (Adravision) system may incorporate BentoML, an open-source platform for deploying machine learning models, and other commercially available software for data handling, storage, and processing.

FIGS. 1(b) and 1(c) are flow charts or flow diagrams illustrating the primary steps, stages, processes, functions, or operations that may be implemented in an embodiment of the disclosed and/or described system for generating dental images and animations for use in educating a patient regarding a dental disease or pathology as part of developing a treatment plan.

As suggested by the figure(s), an embodiment of the disclosed process may include one or more of the following steps, stages, functions, or operations, as illustrated in FIG. 1(b):
Access a patient's dental x-ray (and/or other relevant imagery);
Detect or identify pathological and/or non-pathological features;
Measure bone levels or other relevant features;
Identify anatomical structures;
Apply image processing techniques to illustrate the expected progression of a pathology;
Assess the patient's dental and/or medical situation based on additional information or data from the patient's records or other images; and
Generate one or more personalized images/animations to illustrate potential treatments, the impact of a treatment, or the impact of not pursuing a treatment.

More specifically, as illustrated in FIG. 1(c), in one embodiment, a set of steps, stages, operations, functions, or processes may include:
Capture or access one or more x-rays, intraoral images, or other types of images of a patient's mouth, teeth, and gums (as suggested by step or stage 120);
   This may include classifying the type of x-ray or image (i.e., the image mode and/or subject of the image);
Use one or more image processing techniques (such as a trained model or models) to identify, classify, or otherwise determine the following in the x-rays or images (step or stage 122);
   One or more of the type, location, size, or dimensions of dental pathologies;
   The potential severity of a pathology;
Use one or more image processing techniques (such as a trained model or models) to identify, classify, or otherwise determine non-pathologies (such as previous restorative treatments), anatomical tooth structures, and bone levels (step or stage 124);
   Tooth numbers or other accepted identifier;
   Bone level measurements;
   Anatomical tooth structures;
Generate one or more images or animations illustrating a likely progression over time of an untreated pathology for that patient (step or stage 126);
   This includes utilizing the information obtained from the processing of the images for the specific patient;
   This may include accessing and using other patient-specific health related or dental information;
Generate one or more images or animations illustrating a likely outcome of a proposed treatment plan for that patient (step or stage 128);
   This includes utilizing the information obtained from the processing of the images for the specific patient;
   This typically will also include information regarding the specific treatment plan, its stages, timeline, and components.

In addition to the implementation techniques disclosed and/or described herein, alternative implementations may include one or more of the following:
A model can be trained on existing dental imaging datasets to understand patterns of disease progression such as caries and periapical radiolucency progression;
   Dataset Preparation: Training a generative model for disease progression requires a sufficiently large dataset of dental images representing different stages of the caries or periapical radiolucency progression and how it affects the whole tooth. This dataset is needed for the model to learn the patterns and variations associated with disease progression;
   Labeling and Annotation: Each image in the dataset may need to be labeled or annotated to indicate the specific stage or characteristics of the disease. This labeled data helps the model learn the relationships between visual features and disease progression;
After labeling a dataset, a model such as a Generative Adversarial Networks (GAN) or Variational Autoencoder (VAE) can generate diverse scenarios and stages of a disease, offering a visual narrative of its progression.

Although using a model can result in the generation of a personalized animation of the progression of a disease, it may not be efficient due to one or more of the following:
Generative models, particularly deep learning models like GANs and VAEs, often require large and diverse datasets to accurately capture the complexity of disease progression. If the dataset is limited or biased, it may impact the accuracy of the generated simulations;
Training deep generative models can be computationally intensive, requiring access to one or more GPUs; and
Interpreting the results of generative models, especially in complex medical scenarios, can be challenging. Ensuring that the generated simulations align with known clinical knowledge is important for their practical utility.

Although one or more of the disclosed and/or described embodiments are directed to use of the techniques for purposes of dental diagnosis and education, other potential use cases include one or more of the following:
Diabetic foot ulcers—an animation generated using the techniques disclosed and/or described herein can be used to show that the callus, if left untreated, can lead to complications such as infection and/or gangrene;
Atherosclerosis—an animation can be used to show the buildup of fats, cholesterol, and other substances in/on the artery walls (plaque) causing the arteries to become narrower and less flexible. This plaque buildup blocks blood flow and can eventually rupture and lead to a blood clot; or
Bone fracture healing—an animation can be used to simplify a description of the complex bone fracture healing process, as it typically involves several stages.

In one embodiment, the Adravision system includes a local software application and backend server/platform resident services that utilize computer vision and machine learning models to identify caries, periapical radiolucency, furcation, calculus, and marginal discrepancy in dental radiographs (as non-limiting examples of pathologies), and crown, implant, filling, root canal treatment, and wisdom tooth removal (as non-limiting examples of non-pathologies).

To train some of the disclosed models, thousands of dental radiographs were obtained from several dental clinics around the world. The dataset included 2D dental radiographs namely, bitewing, periapical and panoramic radiographs. Each radiograph was labeled for pathologies (e.g., caries, periapical radiolucency, calculus, furcation, marginal discrepancy) and non-pathologies (e.g., fillings, crowns, root canal treatments, implants, and pontics) by an experienced dentist using an annotation platform. The labels were then reviewed by another dentist, and adjustments were made to improve the accuracy of the labeling, such as adjusting the size of a label, adding or deleting an annotation, or changing the classification of a label.

Labeled images may be pre-processed using resizing and padding techniques to maintain the aspect ratio. The labels were then input to a YOLOv5 object detection and instance segmentation model to detect and outline the pathological and non-pathological features, and a DDR-Net semantic segmentation model to outline the anatomical features.

The YOLOv5 model architecture comprises a backbone and a detection head. The backbone is comprised of a series of convolutional layers and utilizes a sigmoid activation function, along with the integration of skip connections to prevent overfitting and overparameterization. The detection head consists of a YOLO anchor box detector customized to detect the classes of interest.

FIG. 2(h) shows the workflow of the training process. Post processing steps such as filtering bounding box predictions using two thresholds—Intersection over Union (IoU) threshold and confidence threshold—may be used to optimize the results.

To train the bone level measurement model(s), each radiograph within the dataset had the individual teeth cropped out and the labelers were given individual tooth crops to label. The dataset was labeled for the clinical attachment level (CAL) measurement which is the distance from the cementoenamel junction (CEJ) to the level of the bone (BL). Each radiograph was labeled by a dentist or dental nurse/assistant on a labeling platform or by trained professionals on a different platform. Each line segment was reviewed by a dentist and any necessary adjustments were made.

The images were preprocessed, resized, and keypoints translated, then fed into a YOLO pose estimation model (version 8) for keypoint detection. The model was trained from scratch and used bounding boxes to enhance keypoint learning. Post-processing was applied to filter and discard duplicate keypoints, ensuring the correct reconstruction of the CEJ and BL pairs with their corresponding (x,y) coordinates. The workflow followed was similar to the one described for the Pathology and Non-Pathology detection. Post processing was used to optimize the results.

A semantic segmentation model was trained to classify pixels into one of the following categories: enamel, dentin, pulp, or background/others. The training dataset consists of polygons of each category drawn manually by labelers. These polygons are then converted into masks and combined into a pixel-level classification mask. As described, in one embodiment, a state-of-the-art model DDR-Net (as illustrated in FIG. 6) is used as the segmentation model.

Adravision uses image processing techniques to visually represent the natural progression of a pathology polygon, illustrating how a disease can impact anatomical features and cause symptoms. The techniques used to generate the animations should make them look organic and realistic. This is achieved by the overall smooth growth of the pathology (e.g., caries or periapical radiolucency) with some randomness, similar to the gradual creeping of an actual pathology as it grows. The illustrated progression of a pathology should consider the underlying anatomy, e.g., a cavity grows slowly in a tooth's enamel, but very rapidly in its dentin. The animation is designed to accurately represent the growth rates in different parts of a tooth. It also reflects the impact on bone levels and the potential effect on tooth stability. The following sections describe these aspects in greater detail.

### Pathology Progression

The expansion of a pathology begins with a binary raster mask. Initial detection of the pathology (e.g., caries and periapical radiolucency) outline is performed by creating a binary raster mask with a blob of white pixels (a value of 1) indicating the area of the pathology and black pixels (a value of 0) indicating the background (as suggested by FIG. 7(a)). The shape is expanded in a series of expansion cycles. An expansion cycle is a combination of dilation to grow the mass of the blob, and stochastic expansion of border pixels to create realistic non-uniform edges.

### Dilation

This convolution operation is performed by a disk kernel with an anchor point in the center of the kernel (as suggested by FIG. 7(b)). A single dilation operation expands the blob by approximately 3 pixels in all directions.

### Stochastic Expansion

To add some noise to the straight and smooth edges produced by dilation, pixels from a 1-pixel thick outline around the blob (as suggested by FIG. 7(c)) are randomly set to 1. The probability is set to 0.2 by default. This allows for a more realistic expansion of the edge of the pathology outline (as suggested by FIG. 7(d)).

As mentioned, the illustration of the progression of a pathology should consider the underlying anatomy. For example, caries grow in the enamel, dentin, and pulp and not in past restorative treatments such as fillings, crowns, and bridges. Periapical involvements only expand in the bone. To prevent pathologies from growing in all directions (e.g., into space or the bone), instance segmentation masks of tooth anatomy are used.

Pathologies will only expand in regions that overlap with the relevant segmentation masks and at speeds that reflect the actual relative speed of growth. For example, cavities expand in enamel very slowly relative to their expansion in dentin.

The number of expansion cycles per animation frame controls the expansion speed in different regions. As a non-limiting example, the expansion cycle is done once every 7 frames for cavity expansions in enamel and every frame in dentin so that the cavity expands faster in dentin.

FIG. 7(r) is a set of images illustrating a progression of a caries (cavity) that may occur without proper treatment and that may be presented to a patient, in accordance with an embodiment of the disclosure. An implementation in the Python version of OpenCV may be used to perform this operation. FIG. 7(s) is a set of images illustrating a progression of a pathology that may occur without proper treatment and that may be presented to a patient, in accordance with an embodiment of the disclosure. Different color/shade depictions may be used to better illustrate the pathology expansion. For example, when the expansion of the caries reaches the pulp of a tooth, the pulp color may be changed, such as by coloring it brown to indicate an infection of the pulp.

### Periapical Radiolucency & Tooth Instability

Instances of periapical radiolucencies may be indicated by shaded or colored areas around the roots of a tooth. To show the disease progression, the outline of the periapical radiolucency is determined and expanded using the same dilation operation as applied for caries, and only expands in bones and not to other teeth. FIG. 7(g) is a set of images illustrating a timeline or progression of periapical radiolucencies that may occur without proper treatment and that may be presented to a patient, in accordance with an embodiment of the disclosure.

FIG. 7(t) is an image representing an example of periapical radiolucencies that may be presented to a patient, in accordance with an embodiment of the disclosure. When the area of the periapical radiolucency exceeds a certain threshold, the tooth may become unstable due to the lack of bone structure to support it. The outline of the tooth is used to crop and animate the tooth rocking back and forth, as indicated by the white double arrow above it. This potential motion is shown in the set of images in FIG. 7(u).

### Bone Loss Progression

Bone loss is indicated by a drop in bone level. A bone loss progression animation is reliant on anatomical context. FIGS. 7(h-7(m)) are a set of images illustrating the steps involved to create an example of progression of bone loss that may occur without proper treatment and that may be presented to a patient, in accordance with an embodiment of the disclosure. The inputs typically required include:
Bone level points of the mesial (M) and distal (D) surface of every tooth (as suggested by FIG. 7(h)). The key points of bone level on both the mesial (M) and distal (D) sides of each tooth are detected;
   May be detected by a keypoint detector deep learning model;
Connection between adjacent surfaces (as suggested by FIG. 7(i)). The bone level is determined by connecting the mesial key point of one tooth to the distal key point of an adjacent tooth;
   Based on the positions of the teeth and their gaps;
The vertical axes of teeth (as suggested by FIG. 7(j));
   Determined by a tooth orientation deep learning model;
Segmentation mask of bone structure (as suggested by FIG. 7(k));
   Detected by a semantic segmentation deep learning model.
      Using the above inputs, the following values are derived:
Direction of bone loss (as suggested by FIG. 7(l));
   This is determined based on the vertical axes of the teeth (indicated by dashed lines). The bone loss will happen in the opposite direction;
Simulated bone loss (as suggested by FIG. 7(m));
   The areas shown in FIG. 7(m) are cut off when the line shown in FIG. 7(i) is moved down in the direction of the arrows shown in (7(l)). The stimulated bone loss is created by removing the shaded area between the original higher bone level and the now lowered bone level.

### Background Inpainting

As mentioned, a final step requires that the areas of simulated bone loss are replaced with the background. Background refers to the dark empty spaces that are neither teeth or bone/gum. FIGS. 7(n)-7-(q) are images illustrating an example of applying background inpainting in accordance with an embodiment of the disclosure. In one embodiment, this is achieved by:
Get the segmentation mask of the background of the X-ray, indicating area other than teeth and gum (as suggested by FIG. 7(n));
Get the median shade of gray from pixels in areas of the background that is close to the bone level points (intersection between the background space with outline of the teeth and gum as suggested by FIG. 7(o));
Paint the areas of simulated bone loss with this shade of gray;
Blend the areas into the original image at 85% opacity to look more realistic.

After determining the areas of simulated bone loss as shown in FIG. 7(p), they are removed and matched to the background to create the appearance of lost bone, as depicted in FIG. 7(q).

### Treatment Scenario Creation

Once the disease progression animation is created, various treatment scenarios are developed to demonstrate how timely intervention can halt the progression of the disease. A filling might be suggested for a caries that has not yet reached a pulp, or a root canal treatment and a crown will be suggested if caries has reached the pulp.

FIGS. 7(e)-7(g) are images illustrating an example of a dental treatment involving a filling and that may be presented to a patient, in accordance with an embodiment of the disclosure. FIGS. 7(e) to 7(g) illustrate how a cavity that has not yet reached the pulp can be halted through use of a filling. 7(e) depicts caries that has not yet reached the pulp, 7(f) shows a caries removed, and 7(g) shows how the empty space may be filled with a filling treatment.

FIGS. 7(v)-7(cc) are images illustrating a root canal crown procedure that may be presented to a patient, in accordance with an embodiment of the disclosure. FIGS. 7(v) to 7(cc) are figures/images showing how late intervention will result in a root canal, post and core, and a crown treatment. The process is such that the area of the infection is removed and an access to pulp is created to do the root canal treatment (FIG. 7(w)).

After the root canal treatment is done (FIG. 7(x)), a post or posts are placed, and core is built to replace the tooth structure (FIG. 7(y) and FIG. 7(z)). The tooth/core is then shaved to a cone shape and a crown in the shape of the original tooth structure is placed (FIG. 7(aa) and FIG. 7(bb)). The infection at the tip of the root will resolve and disappear anywhere between 4 months to 4 years post root canal treatment (FIG. 7(cc)).

FIG. 2 is a diagram illustrating elements or components that may be present in a computing device or system configured to implement a method, process, function, or operation in accordance with an embodiment of the system and methods disclosed and/or described herein. As shown in the figure and as mentioned, in some embodiments, the system and methods may be implemented in the form of an apparatus, platform, device, or server that includes a processing element and set of computer-executable instructions. The executable instructions may be stored in (or on) a non-transitory memory or data storage element and be part of a software application arranged into a software architecture.

In general, an embodiment may be implemented using a set of software instructions that are designed to be executed by a suitably programmed processing element (such as a GPU, CPU, TPU, QPU, state machine, microprocessor, processor, co-processor, or controller, as non-limiting examples). In a complex application or system such instructions are typically arranged into “modules” (or sub-modules) with each such module typically performing a specific task, process, function, or operation. The entire set of modules may be controlled or coordinated in their operation by an operating system (OS) or other form of organizational platform.

Each application module or sub-module may correspond to a particular function, method, process, or operation that is implemented by the module or submodule. Such function, method, process, or operation may include those used to implement one or more aspects of the disclosed and/or described systems and methods.

The application modules and/or sub-modules may include a suitable computer-executable code or set of instructions (e.g., as would be executed by a suitably programmed processor, microprocessor, co-processor, or CPU, as examples), such as computer-executable code corresponding to a programming language. For example, programming language source code may be compiled into computer-executable code. Alternatively, or in addition, the programming language may be an interpreted programming language such as a scripting language.

Modules (or sub-modules) may contain one or more sets of instructions for performing a method or function described with reference to the Figures, and the descriptions or disclosure of the functions and operations provided in this specification. The modules may include those illustrated but may also include a greater number or fewer number than those illustrated. As mentioned, each module may contain a set of computer-executable instructions. The set of instructions may be executed by a programmed processor contained in one or more of a server, client device, network element, system, platform, or other component.

A module (or sub-module) may contain instructions that are executed by a processor contained in more than one of a server, client device, network element, system, platform, or other component. Thus, in some embodiments, a plurality of electronic processors, with each being part of a separate server, client device, network element, system, or platform may be responsible for executing all or a portion of the software instructions contained in an illustrated module. Although FIG. 2(a) illustrates a set of modules which taken together perform multiple functions or operations, these functions or operations may be performed by different devices or system components, with certain of the modules (or instructions contained in those modules) being associated with those devices or system components.

As shown in FIG. 2(a), system 200 may represent a server or other form of computing or data processing system, server, platform, or device. Modules 202 each contain a set of executable instructions, where when the set of instructions is executed by a suitable electronic processor or processors (such as that indicated in the figure by “Physical Processor(s) 230”), system (or server, platform, or device) 200 operates to perform a specific process, operation, function, or method. Modules 202 are stored in a non-transitory memory 220, which typically includes an Operating System module 204 that contains instructions used (among other functions) to access and control the execution of the instructions contained in other modules.

The modules 202 stored in memory 220 are accessed for purposes of transferring data and executing instructions by use of a “bus” or communications line 219, which also serves to permit processor(s) 230 to communicate with the modules for purposes of accessing and executing a set of instructions. Bus or communications line 219 also permits processor(s) 230 to interact with other elements of system 200, such as input or output devices 222, communications elements 224 for exchanging data and information with devices external to system 200, and additional memory devices 226.

In some embodiments, the modules may comprise computer-executable software instructions that when executed by one or more electronic processors or co-processors cause the processors or co-processors (or a system, device, or apparatus containing the processors or co-processors) to perform one or more of the following steps, stages, functions, operations, or processes:
Capture or access one or more x-rays, intraoral images, or other types of images of a patient's mouth, teeth, and gums (as suggested by module 206);
Use one or more image processing techniques (such as a trained model or models) to identify (or determine) the following in the x-rays or images (as suggested by module 208);
   One or more of the type, location, size, or dimensions of dental pathologies and non-pathologies (such as previous restorative treatments);
      The potential severity of a pathology;
   Tooth numbers or other identifier;
   Measurement of bone levels;
   Anatomical tooth structures;
Generate one or more images or animations illustrating a likely progression over time of an untreated pathology for that patient (as suggested by module 210);
   This includes utilizing the information obtained from the processing of the images for the specific patient;
   This may include accessing and using other patient-specific health/dental information; and
Generate one or more images or animations illustrating a likely outcome of a proposed treatment plan for that patient (as suggested by module 212);
   This includes utilizing the information obtained from the processing of the images for the specific patient;
   This typically will also include information regarding the specific treatment plan, its stages, timeline, and components.

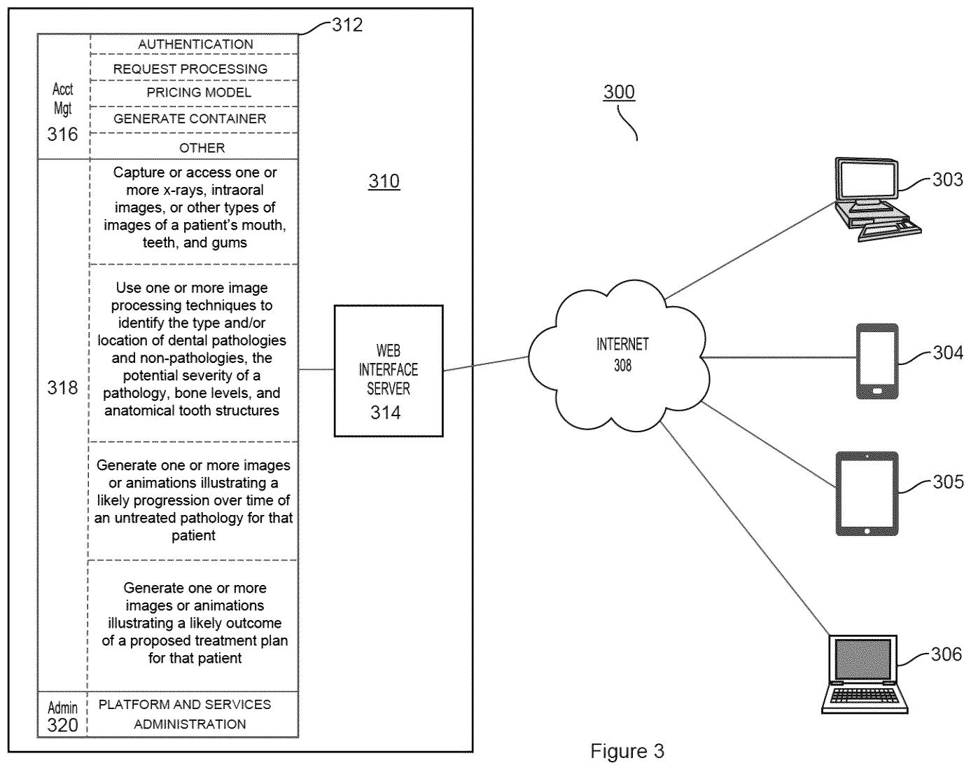

In some embodiments, the functionality and services provided by the system and methods disclosed and/or described herein may be made available to multiple users by accessing an account maintained by a server or service platform. Such a server or service platform may be termed a form of Software-as-a-Service (Saas). FIGS. 3-5 are diagrams illustrating a deployment of the system and methods disclosed and/or described herein for assisting dental service providers and educating patients by generating dental images and animations to assist in understanding a dental disease or pathology as part of developing a treatment plan, in accordance with some embodiments.

In some embodiments, the system or service(s) disclosed and/or described herein may be implemented as micro-services, processes, workflows, or functions performed in response to a user request (where in this situation, a “user” may be a dental service provider or other process performed by the platform or system). The micro-services, processes, workflows, or functions may be performed by a server, data processing element, platform, or system.

In some embodiments, the services may be provided by a service platform located “in the cloud”. In such embodiments, the platform is accessible through APIs and SDKs. The disclosed and/or described processing and services may be provided as micro-services within the platform for each of multiple users. The interfaces to the micro-services may be defined by REST and GraphQL endpoints. An administrative console may allow users or an administrator to securely access the underlying request and response data, manage accounts and access, and in some cases, modify the processing workflow or configuration.

Although in some embodiments, a platform or system of the type illustrated in FIGS. 3-5 may be operated by a 3^{rd} party provider to provide a specific set of business-related applications, in other embodiments, the platform may be operated by a provider and a different business may provide the applications or services for users through the platform.

FIG. 3 is a diagram illustrating a system 300 in which an embodiment of the disclosure may be implemented or through which an embodiment of the services disclosed and/or described herein may be accessed. In accordance with the advantages of an application service provider (ASP) hosted business service system (such as a multi-tenant data processing platform), users of the services may comprise individuals (such as dental service providers), businesses (such as an insurance company), or organizations (such as a group of dentists), as non-limiting examples.

A user may access the services using a suitable client, including but not limited to desktop computers, laptop computers, tablet computers, or smartphones. Users interface with the service platform across the Internet 308 or another suitable communications network or combination of networks. Examples of suitable client devices include desktop computers 303, smartphones 304, tablet computers 305, or laptop computers 306.

System 310, which may be hosted by a third party, may include a set of services 312 and a web interface server 314, coupled as shown in FIG. 3. It is to be appreciated that either or both services 312 and web interface server 314 may be implemented on one or more different hardware systems and components, even though represented as singular units in FIG. 3. Services 312 may include one or more functions or operations for the processing and interpretation of dental images, the generation of images or animations to suggest the progression of an untreated pathology, and the generation of images or animations to suggest the progression of a pathology under a specific treatment plan, as non-limiting examples.

In some embodiments, the set of services or applications available to a user may include one or more that perform the functions and methods disclosed in the specification and/or described with reference to the figures. As examples, in some embodiments, the set of applications, functions, operations or services made available through the platform or system 310 may include:
account management services 316, such as
   a process or service to authenticate a person wishing to access the services/applications available through the platform (such as credentials, proof of purchase, or verification that the customer has been authorized by a company to use the services);
   a process or service to generate a container or instantiation of the services, methodology, applications, functions, and operations disclosed and/or described, where the instantiation may be customized for a particular user or company; and
   other forms of account management services;
a set 318 of data processing services, applications, or functionality, such as a process or service to:
   Capture or access one or more x-rays, intraoral images, or other types of images of a patient's mouth, teeth, and gums;
   Use one or more image processing techniques (such as a trained model or models) to identify or determine the following in the x-rays or images;
      One or more of the type, location, size, or dimensions of dental pathologies and non-pathologies (such as previous restorative treatments);
      The potential severity of a pathology;
      Tooth numbers or other identifier;
      Measurement of bone levels;
      Anatomical tooth structures;
   Generate one or more images or animations illustrating a likely progression over time of an untreated pathology for that patient;
      This includes utilizing the information obtained from the processing of the images for the specific patient;
      This may include accessing and using other patient-specific health/dental information; and
   Generate one or more images or animations illustrating a likely outcome of a proposed treatment plan for that patient;
      This includes utilizing the information obtained from the processing of the images for the specific patient;
      This typically will also include information regarding the specific treatment plan, its stages, timeline, and components;
administrative services 320, such as
   a process or services to enable the provider of the data processing services and/or the platform to administer and configure the processes and services provided to users.

The platform or system shown in FIG. 3 may be hosted on a distributed computing system made up of at least one, but typically multiple, “servers.” A server is a physical computer dedicated to providing data storage and an execution environment for one or more software applications or services intended to serve the needs of the users of other computers that are in data communication with the server, for instance via a public network such as the Internet. The server, and the services it provides, may be referred to as the “host” and the remote computers, and the software applications running on the remote computers being served may be referred to as “clients.” Depending on the computing service(s) that a server offers it could be referred to as a database server, data storage server, file server, mail server, print server, or web server.

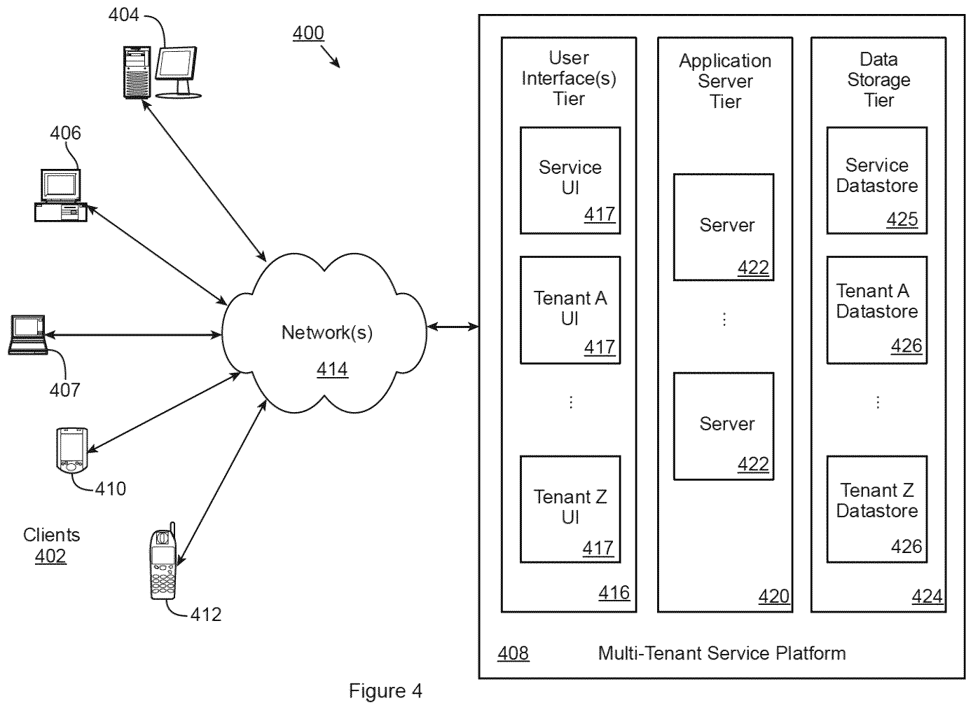

FIG. 4 is a diagram illustrating elements or components of an example operating environment 400 in which an embodiment of the disclosure may be implemented. As shown, a variety of clients 402 incorporating and/or incorporated into a variety of computing devices may communicate with a multi-tenant service platform 408 through one or more networks 414. For example, a client may incorporate and/or be incorporated into a client application (e.g., software) implemented at least in part by one or more of the computing devices. Examples of suitable computing devices include personal computers, server computers 404, desktop computers 406, laptop computers 407, notebook computers, tablet computers or personal digital assistants (PDAs) 410, smart phones 412, cell phones, and consumer electronic devices incorporating one or more computing device components, such as one or more electronic processors, microprocessors, central processing units (CPU), or controllers. Examples of suitable networks 414 include networks utilizing wired and/or wireless communication technologies and networks operating in accordance with any suitable networking and/or communication protocol (e.g., the Internet).

The distributed computing service/platform (which may also be referred to as a multi-tenant data processing platform) 408 may include multiple processing tiers, including a user interface tier 416, an application server tier 420, and a data storage tier 424. The user interface tier 416 may maintain multiple user interfaces 417, including graphical user interfaces and/or web-based interfaces. The user interfaces may include a default user interface for the service to provide access to applications and data for a user or “tenant” of the service (depicted as “Service UI” in the figure), as well as one or more user interfaces that have been specialized/customized in accordance with user specific requirements (e.g., represented by “Tenant A UI”, . . . , “Tenant Z UI” in the figure, and which may be accessed via one or more APIs).

The default user interface may include user interface components enabling a tenant to administer the tenant's access to and use of the functions and capabilities provided by the service platform. This may include accessing tenant data, launching an instantiation of a specific application, causing the execution of specific data processing operations, as an example.

Each application server 422 or processing tier 420 shown in the figure may be implemented with a set of computers and/or components including computer servers and processors, and may perform various functions, methods, processes, or operations as determined by the execution of a software application or set of instructions. The data storage tier 424 may include one or more data stores, which may include a Service Data store 425 and one or more Tenant Data stores 426. Data stores may be implemented with a suitable data storage technology, including structured query language (SQL) based relational database management systems (RDBMS).

Service Platform 408 may be multi-tenant and may be operated by an entity to provide multiple tenants with a set of business-related or other data processing applications, data storage, and functionality. For example, the applications and functionality may include providing web-based access to the functionality used by a business to provide services to end-users, thereby allowing a user with a browser and an Internet or intranet connection to view, enter, process, or modify certain types of information. Such functions or applications are typically implemented by one or more modules of software code/instructions that are maintained on and executed by one or more servers 422 that are part of the platform's Application Server Tier 420. As noted with regards to FIG. 3, the platform system shown in FIG. 4 may be hosted on a distributed computing system made up of at least one, but typically multiple, “servers.”

As mentioned, rather than build and maintain such a platform or system themselves, a business may utilize systems provided by a third party. A third party may implement a business system/platform as described above in the context of a multi-tenant platform, where individual instantiations of a business' data processing workflow (such as the image processing and generation of animations disclosed and/or described herein) are provided to users, with each user or group of users representing a tenant of the platform. One advantage to such multi-tenant platforms is the ability for each tenant to customize their instantiation of the data processing workflow to that tenant's specific business needs or operational methods. In some cases, each tenant may be a business or entity that uses the multi-tenant platform to provide services and functionality to multiple end-users.

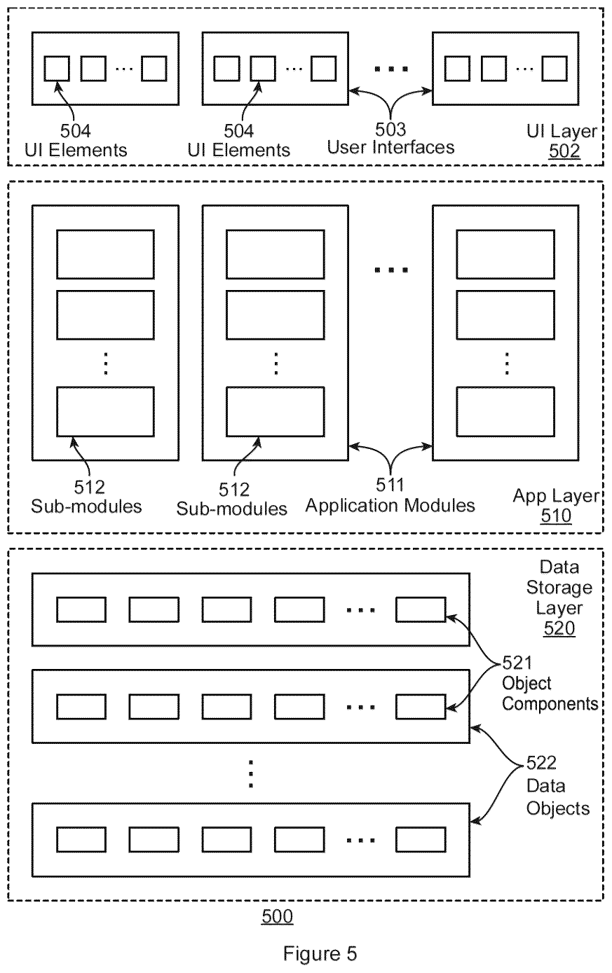

FIG. 5 is a diagram illustrating additional details of the elements or components of the multi-tenant distributed computing service platform of FIG. 4, in which an embodiment of the disclosure may be implemented. The software architecture shown in FIG. 5 represents an example of an architecture which may be used to implement an embodiment of the disclosure. In general, an embodiment may be implemented using a set of software instructions that are executed by a suitably programmed processing element (such as a CPU, GPU, microprocessor, processor, co-processor, or controller, as non-limiting examples). In a complex system such instructions are typically arranged into “modules” with each such module performing a specific task, process, function, or operation. The entire set of modules may be controlled or coordinated in their operation by an operating system (OS) or other form of organizational platform.

As noted, FIG. 5 is a diagram illustrating additional details of the elements or components 500 of a multi-tenant distributed computing service platform, in which an embodiment of the disclosure may be implemented. The example architecture includes a user interface layer or tier 502 having one or more user interfaces 503. Examples of such user interfaces include graphical user interfaces and application programming interfaces (APIs). Each user interface may include one or more user interface (UI) elements 504.

For example, users may interact with user interface elements to access functionality and/or data provided by application and/or data storage layers of the example architecture. Examples of graphical user interface elements include buttons, menus, checkboxes, drop-down lists, scrollbars, sliders, spinners, text boxes, icons, labels, progress bars, status bars, toolbars, windows, hyperlinks, and dialog boxes. Application programming interfaces may be local or remote and may include interface elements such as parameterized procedure calls, programmatic objects, and messaging protocols.

The application layer 510 may include one or more application modules 511, each having one or more sub-modules 512. Each application module 511 or sub-module 512 may correspond to a function, method, process, or operation that is implemented by the module or sub-module (e.g., a function or process related to providing data processing and services to a user of the platform). Such function, method, process, or operation may include those used to implement one or more aspects of the disclosed system and methods, such as for one or more of the processes or functions disclosed herein and/or described with reference to the Figures:
Capture or access one or more x-rays, intraoral images, or other types of images of a patient's mouth, teeth, and gums;
Use one or more image processing techniques (such as a trained model or models) to identify or determine the following in the x-rays or images;
   One or more of the type, location, size, or dimensions of dental pathologies and non-pathologies (such as previous restorative treatments);
   The potential severity of a pathology;
   Tooth numbers or other identifier;
   Measurement of bone levels;
   Anatomical tooth structures;
Generate one or more images or animations illustrating a likely progression over time of an untreated pathology for that patient;
   This includes utilizing the information obtained from the processing of the images for the specific patient;
   This may include accessing and using other patient-specific health/dental information; and
Generate one or more images or animations illustrating a likely outcome of a proposed treatment plan for that patient;
   This includes utilizing the information obtained from the processing of the images for the specific patient;
   This typically will also include information regarding the specific treatment plan, its stages, timeline, and components.

The application modules and/or submodules may include any suitable computer-executable code or set of instructions (e.g., as would be executed by a suitably programmed processor, microprocessor, or CPU), such as computer-executable code corresponding to a programming language. For example, programming language source code may be compiled into computer-executable code. Alternatively, or in addition, the programming language may be an interpreted programming language such as a scripting language. Each application server (e.g., as represented by element 422 of FIG. 4) may include each application module. Alternatively, different application servers may include different sets of application modules. Such sets may be disjoint or overlapping.

The data storage layer 520 may include one or more data objects 522 each having one or more data object components 521, such as attributes and/or behaviors. For example, the data objects may correspond to tables of a relational database, and the data object components may correspond to columns or fields of such tables. Alternatively, or in addition, the data objects may correspond to data records having fields and associated services. Alternatively, or in addition, the data objects may correspond to persistent instances of programmatic data objects, such as structures and classes. Each data store in the data storage layer may include each data object. Alternatively, different data stores may include different sets of data objects. Such sets may be disjoint or overlapping.

Note that the example computing environments depicted in FIGS. 3-5 are not intended to be limiting examples. Further environments in which an embodiment of the invention may be implemented in whole or in part include devices (including mobile devices), software applications, systems, apparatuses, networks, SaaS platforms, IaaS (infrastructure-as-a-service) platforms, or other configurable components that may be used by multiple users for data entry, data processing, application execution, or data review.

This disclosure includes the following embodiments or clauses:
1. A method of providing a dental service to a patient, comprising:
obtaining one or more x-rays, intraoral images, or other images of a patient's mouth, teeth, and gums;
using one or more trained image processing models to identify a type, location, size, or dimension of a dental pathology or non-pathology in the x-rays, intraoral images, or other images;
generating one or more images or animations illustrating a likely progression over time of the dental pathology for that patient if not treated; and
generating one or more images or animations illustrating a likely outcome of a proposed treatment plan for the dental pathology for that patient.
2. The method of clause 1, wherein the one or more trained image processing models further comprise a model or models that operate to:
classify the type of x-ray or image;
determine the potential severity of a pathology;
identify a tooth number or identifier associated with each tooth;
determine a measurement of bone levels; and
identify one or more anatomical tooth structures.
3. The method of clause 2, wherein the dental pathology is one or more of caries, periapical radiolucency, calculus, and furcation, and the non-pathology is one or more of a previous restorative treatment, wisdom tooth removal, and inferior alveolar nerve treatment.
4. The method of clause 2, wherein the anatomical tooth structure is one or more of dentin, enamel, and pulp.
5. The method of clause 1, further comprising accessing a dental or health record of the patient and using information in the dental or health record as part of assessing the patient's dental condition or planning a treatment.
6. A system for assisting a dentist to provide a dental service to a patient, comprising:
a non-transitory computer-readable medium including a set of computer-executable instructions;
one or more electronic processors configured to execute the set of computer-executable instructions, wherein when executed, the instructions cause the one or more electronic processors to
   obtain one or more x-rays, intraoral images, or other images of a patient's mouth, teeth, and gums;
   use one or more trained image processing models to identify a type, location, size, or dimension of a dental pathology or non-pathology in the x-rays, intraoral images, or other images;
   generate one or more images or animations illustrating a likely progression over time of the dental pathology for that patient if not treated; and
   generate one or more images or animations illustrating a likely outcome of a proposed treatment plan for the dental pathology for that patient.
7. One or more non-transitory computer-readable media including a set of computer-executable instructions that when executed by one or more programmed electronic processors, cause the processors to:
obtain one or more x-rays, intraoral images, or other images of a patient's mouth, teeth, and gums;
use one or more trained image processing models to identify a type, location, size, or dimension of a dental pathology or non-pathology in the x-rays, intraoral images, or other images;
generate one or more images or animations illustrating a likely progression over time of the dental pathology for that patient if not treated; and
generate one or more images or animations illustrating a likely outcome of a proposed treatment plan for the dental pathology for that patient.

Embodiments of the disclosure may be implemented in the form of control logic using computer software in a modular or integrated manner. Based on the disclosure and teachings provided herein, a person of ordinary skill in the art will recognize other ways and/or methods to implement an embodiment using hardware, software, or a combination of hardware and software.

In some embodiments, certain of the methods, models, processes, or functions disclosed herein may be embodied in the form of a trained neural network or other form of model derived from a machine learning algorithm. The neural network or model may be implemented by the execution of a set of computer-executable instructions and/or represented as a data structure. The instructions may be stored in (or on) a non-transitory computer-readable medium and executed by a programmed processor or processing element. The set of instructions may be conveyed to a user through a transfer of instructions or an application that executes a set of instructions over a network (e.g., the Internet). The set of instructions or an application may be utilized by an end-user through access to a SaaS platform, self-hosted software, on-premise software, or a service provided through a remote platform.

In general terms, a neural network may be viewed as a system of interconnected artificial “neurons” or nodes that exchange messages between each other. The connections have numeric weights that are “tuned” during a training process, so that a properly trained network will respond correctly when presented with an image, pattern, or set of data. In this characterization, the network consists of multiple layers of feature-detecting “neurons”, where each layer has neurons that respond to different combinations of inputs from the previous layers.

Training of a network is performed using a “labeled” dataset of inputs in an assortment of representative input patterns (or datasets) that are associated with their intended output response. Training uses general-purpose methods to iteratively determine the weights for intermediate and final feature neurons. In terms of a computational model, each neuron calculates the dot product of inputs and weights, adds a bias, and applies a non-linear trigger or activation function (for example, using a sigmoid response function).

Machine learning (ML) is used to analyze data and assist in making decisions in multiple industries. To benefit from using machine learning, a machine learning algorithm is applied to a set of training data and labels to generate a “model” which represents what the application of the algorithm has “learned” from the training data. Each element (or example) in the form of one or more parameters, variables, characteristics, or “features” of the set of training data is associated with a label or annotation that defines how the element should be classified by the trained model. A machine learning model can predict or infer an outcome based on the training data and labels and be used as part of decision process. When trained, the model will operate on a new element of input data to generate the correct label or classification as an output.

Any of the software components, processes or functions described in this application may be implemented as software code to be executed by a processor using any suitable computer language such as Python, Java, Javascript, C++, or Perl using procedural, functional, object-oriented, or other techniques. The software code may be stored as a series of instructions, or commands in (or on) a non-transitory computer-readable medium, such as a random-access memory (RAM), a read only memory (ROM), a magnetic medium such as a hard-drive, or an optical medium such as a CD-ROM. In this context, a non-transitory computer-readable medium is almost any medium suitable for the storage of data or an instruction set aside from a transitory waveform. Any such computer readable medium may reside on or within a single computational apparatus and may be present on or within different computational apparatuses within a system or network.

According to one example implementation, the term processing element or processor, as used herein, may be a central processing unit (CPU), or conceptualized as a CPU (such as a virtual machine). In this example implementation, the CPU or a device in which the CPU is incorporated may be coupled, connected, and/or in communication with one or more peripheral devices, such as display. In another example implementation, the processing element or processor may be incorporated into a mobile computing device, such as a smartphone or tablet computer.

The non-transitory computer-readable storage medium referred to herein may include a number of physical drive units, such as a redundant array of independent disks (RAID), a flash memory, a USB flash drive, an external hard disk drive, thumb drive, pen drive, key drive, a High-Density Digital Versatile Disc (HD-DV D) optical disc drive, an internal hard disk drive, a Blu-Ray optical disc drive, or a Holographic Digital Data Storage (HDDS) optical disc drive, synchronous dynamic random access memory (SDRAM), or similar devices or other forms of memories based on similar technologies. Such computer-readable storage media allow the processing element or processor to access computer-executable process steps, application programs and the like, stored on removable and non-removable memory media, to off-load data from a device or to upload data to a device. As mentioned, with regards to the embodiments described herein, a non-transitory computer-readable medium may include almost any structure, technology or method apart from a transitory waveform or similar medium.

Certain implementations of the disclosed technology are described herein with reference to block diagrams of systems, and/or to flowcharts or flow diagrams of functions, operations, processes, or methods. It will be understood that one or more blocks of the block diagrams, or one or more stages or steps of the flowcharts or flow diagrams, and combinations of blocks in the block diagrams and stages or steps of the flowcharts or flow diagrams, respectively, may be implemented by computer-executable program instructions. Note that in some embodiments, one or more of the blocks, or stages or steps may not necessarily need to be performed in the order presented or may not necessarily need to be performed at all.

These computer-executable program instructions may be loaded onto a general-purpose computer, a special purpose computer, a processor, or other programmable data processing apparatus to produce a specific example of a machine, such that the instructions that are executed by the computer, processor, or other programmable data processing apparatus create means for implementing one or more of the functions, operations, processes, or methods described herein. These computer program instructions may also be stored in a computer-readable memory that may direct a computer or other programmable data processing apparatus to function in a specific manner, such that the instructions stored in the computer-readable memory produce an article of manufacture including instruction means that implement one or more of the functions, operations, processes, or methods disclosed or described herein.

While certain implementations of the disclosed technology have been described in connection with what is presently considered to be the most practical and various implementations, it is to be understood that the disclosed technology is not to be limited to the disclosed implementations. Instead, the disclosed implementations are intended to cover various modifications and equivalent arrangements included within the scope of the appended claims. Although specific terms are employed herein, they are used in a generic and descriptive sense only and not for purposes of limitation.

This written description uses examples to disclose certain implementations of the disclosed technology, and to enable any person skilled in the art to practice certain implementations of the disclosed technology, including making and using any devices or systems and performing any incorporated methods. The patentable scope of certain implementations of the disclosed technology is defined in the claims, and may include other examples that occur to those skilled in the art. Such other examples are intended to be within the scope of the claims if they have structural and/or functional elements that do not differ from the literal language of the claims, or if they include structural and/or functional elements with insubstantial differences from the literal language of the claims.

All references, including publications, patent applications, and patents, cited herein are hereby incorporated by reference to the same extent as if each reference were individually and specifically indicated to be incorporated by reference and/or were set forth in its entirety herein.

The use of the terms “a” and “an” and “the” and similar referents in the specification and in the following claims are to be construed to cover both the singular and the plural, unless otherwise indicated herein or clearly contradicted by context. The terms “having,” “including,” “containing” and similar referents in the specification and in the following claims are to be construed as open-ended terms (e.g., meaning “including, but not limited to,”) unless otherwise noted. Recitation of ranges of values herein are merely intended to serve as a shorthand method of referring individually to each separate value inclusively falling within the range, unless otherwise indicated herein, and each separate value is incorporated into the specification as if it were individually recited herein. All methods described herein may be performed in any suitable order unless otherwise indicated herein or clearly contradicted by context. The use of all examples, or exemplary language (e.g., “such as”) provided herein, is intended merely to better illuminate embodiments of the invention and does not pose a limitation to the scope of the invention unless otherwise claimed. No language in the specification should be construed as indicating any non-claimed element as essential to each embodiment of the present invention.

As used herein (i.e., the claims, figures, and specification), the term “or” is used inclusively to refer to items in the alternative and in combination.

Different arrangements of the components depicted in the drawings or described above, as well as components and steps not shown or described are possible. Similarly, some features and sub-combinations are useful and may be employed without reference to other features and sub-combinations. Embodiments of the invention have been described for illustrative and not restrictive purposes, and alternative embodiments will become apparent to readers of this patent. Accordingly, the present invention is not limited to the embodiments described above or depicted in the drawings, and various embodiments and modifications may be made without departing from the scope of the claims below.

What is claimed is:

1. A method of providing a dental service to a patient, comprising:
obtaining one or more x-rays, intraoral images, or other images of a patient's mouth, teeth, and gums;
using one or more trained image processing models to identify a type, location, size, or dimension of a dental pathology or non-pathology in the x-rays, intraoral images, or other images;
generating one or more images or animations illustrating a likely progression over time of the dental pathology for that patient if not treated; and
generating one or more images or animations illustrating a likely outcome of a proposed treatment plan for the dental pathology for that patient.

2. The method of claim 1, wherein the one or more trained image processing models further comprise a model or models that operate to:
classify the type of x-ray or image;
determine the potential severity of a pathology;
identify a tooth number or identifier associated with each tooth;
determine a measurement of bone levels; and
identify one or more anatomical tooth structures.

3. The method of claim 2, wherein the dental pathology is one or more of caries, periapical radiolucency, calculus, and furcation, and the non-pathology is one or more of a previous restorative treatment, wisdom tooth removal, and inferior alveolar nerve treatment.

4. The method of claim 2, wherein the anatomical tooth structure is one or more of dentin, enamel, and pulp.

5. The method of claim 1, further comprising accessing a dental or health record of the patient and using information in the dental or health record as part of assessing the patient's dental condition or planning a treatment.

6. The method of claim 1, further comprising initially obtaining the x-rays, intraoral images, or other images in a dental service provider's office and providing them to a remote server or platform, the remote server or platform hosting a set of services that include using the one or more trained image processing models and generating the images or animations.

7. The method of claim 6, further comprising receiving the generated images or animations from the remote server or platform at an application or workstation located in the dental service provider's office.

8. A system for assisting a dentist to provide a dental service to a patient, comprising:
a non-transitory computer-readable medium including a set of computer-executable instructions;
one or more electronic processors configured to execute the set of computer-executable instructions, wherein when executed, the instructions cause the one or more electronic processors to
obtain one or more x-rays, intraoral images, or other images of a patient's mouth, teeth, and gums;
use one or more trained image processing models to identify a type, location, size, or dimension of a dental pathology or non-pathology in the x-rays, intraoral images, or other images;
generate one or more images or animations illustrating a likely progression over time of the dental pathology for that patient if not treated; and
generate one or more images or animations illustrating a likely outcome of a proposed treatment plan for the dental pathology for that patient.

9. The system of claim 8, wherein the one or more trained image processing models further comprise a model or models that operate to:
classify the type of x-ray or image;
determine the potential severity of a pathology;
identify a tooth number or identifier associated with each tooth;
determine a measurement of bone levels; and
identify one or more anatomical tooth structures.

10. The system of claim 9, wherein the dental pathology is one or more of caries, periapical radiolucency, calculus, and furcation, and the non-pathology is one or more of a previous restorative treatment, wisdom tooth removal, and inferior alveolar nerve treatment.

11. The system of claim 9, wherein the anatomical tooth structure is one or more of dentin, enamel, and pulp.

12. The system of claim 8, wherein when executed, the instructions cause the one or more electronic processors to access a dental or health record of the patient and use information in the dental or health record as part of assessing the patient's dental condition or planning a treatment.

13. The system of claim 8, wherein when executed, the instructions cause the x-rays, intraoral images, or other images to initially be obtained in a dental service provider's office and provided to a remote server or platform, the remote server or platform hosting a set of services that include using the one or more trained image processing models and generating the images or animations.

14. The system of claim 13, wherein when executed, the instructions cause the generated images or animations to be received at an application or workstation located in the dental service provider's office.

15. One or more non-transitory computer-readable media including a set of computer-executable instructions that when executed by one or more programmed electronic processors, cause the processors to:
obtain one or more x-rays, intraoral images, or other images of a patient's mouth, teeth, and gums;
use one or more trained image processing models to identify a type, location, size, or dimension of a dental pathology or non-pathology in the x-rays, intraoral images, or other images;
generate one or more images or animations illustrating a likely progression over time of the dental pathology for that patient if not treated; and
generate one or more images or animations illustrating a likely outcome of a proposed treatment plan for the dental pathology for that patient.

16. The one or more non-transitory computer-readable media of claim 15, wherein the one or more trained image processing models further comprise a model or models that operate to:
classify the type of x-ray or image;
determine the potential severity of a pathology;
identify a tooth number or identifier associated with each tooth;
determine a measurement of bone levels; and
identify one or more anatomical tooth structures.

17. The one or more non-transitory computer-readable media system of claim 16, wherein the dental pathology is one or more of caries, periapical radiolucency, calculus, and furcation, and the non-pathology is one or more of a previous restorative treatment, wisdom tooth removal, and inferior alveolar nerve treatment.

18. The one or more non-transitory computer-readable media system of claim 16, wherein the anatomical tooth structure is one or more of dentin, enamel, and pulp.

19. The one or more non-transitory computer-readable media system of claim 15, wherein when executed, the instructions cause the one or more electronic processors to access a dental or health record of the patient and use information in the dental or health record as part of assessing the patient's dental condition or planning a treatment.

20. The one or more non-transitory computer-readable media system of claim 15, wherein when executed, the instructions cause the x-rays, intraoral images, or other images to initially be obtained in a dental service provider's office and provided to a remote server or platform, the remote server or platform hosting a set of services that include using the one or more trained image processing models and generating the images or animations, and further cause the generated images or animations to be received at an application or workstation located in the dental service provider's office.
